(19) United States
(12) Patent Application Publication
(10) Pub. No.: US 2017/0258648 A1
(43) Pub. Date: Sep. 14, 2017

(54) THREE-DIMENSIONAL MATERIALS HAVING APERTURES

(71) Applicant: The Procter & Gamble Company, Cincinnati, OH (US)

(72) Inventors: Rodrigo ROSATI, Frankfurt Am Main (DE); Jill Marlene ORR, Liberty Township, OH (US)

(21) Appl. No.: 15/454,008

(22) Filed: Mar. 9, 2017

Related U.S. Application Data
(60) Provisional application No. 62/306,877, filed on Mar. 11, 2016.

(51) Int. Cl. A61F 13/511; A61F 13/536; A61F 13/51; A61F 13/539

(52) U.S. Cl. CPC A61F 13/51104; A61F 13/539; A61F 13/536; A61F 13/52; A61F 2013/51361

(57) ABSTRACT

An absorbent article comprising a liquid permeable nonwoven topsheet, a nonwoven second material that is a separate material from the topsheet, a liquid impermeable backsheet, and an absorbent core positioned intermediate the second material and the backsheet. The second material is positioned intermediate the topsheet and absorbent core. The topsheet is nested with the second material to form a nested laminate comprising a plurality of recesses and a plurality of raised areas. Voids are defined in the raised areas under the second material. A first aperture is formed in a substantially central location of the raised areas. The recesses each form a base positioned most distal from the substantially central locations of the raised areas. A second aperture is formed in the bases of the recesses. The first and second apertures extend through both the topsheet and second material.

### CROSS REFERENCE TO RELATED APPLICATION

This application claims the benefit, under 35 U.S.C. §119(e), to U.S. Provisional Patent Application No. 62/306,877 filed on Mar. 11, 2016, the entire disclosure of which is hereby incorporated by reference.

### FIELD

The present disclosure is directed to three-dimensional materials having apertures. The present disclosure is also directed to absorbent articles comprising three-dimensional nonwoven materials having apertures.

### BACKGROUND

Absorbent articles typically comprise a topsheet, a backsheet, and an absorbent core disposed between the topsheet and the backsheet. The absorbent article may also comprise an acquisition layer that temporarily stores liquid bodily exudates received from the topsheet and an optional distribution layer that transfers and distributes the liquid bodily exudates from the acquisition layer to the absorbent core.

Many absorbent articles, including diapers, rely on capillary action to achieve fluid acquisition and wicking of fluid away from the skin of a wearer. The structure of absorbent articles generally results in a configuration in which there is a higher capillary pressure in the bottom layer and a lower capillary pressure in the top layer. Some absorbent articles also comprise textured and/or apertured topsheets to improve fluid handling properties. However, absorbent articles made from these materials are typically less soft. In addition, the fluid handling properties of these materials may be somewhat limited, particularly when handling both urine and viscous body fluids such as a runny bowel movement (“BM”).

Thus, there is a need for improved materials for use in absorbent articles. In particular, a need exists for improved nonwoven materials or laminates of nonwoven materials or laminates comprising nonwoven materials that have improved dryness, and have improved absorbency and retention of BM and other bodily fluids, and reduced run-off. In particular, a need exists for improved nonwoven materials having three-dimensional features and apertures formed therein to provide improved absorbency and retention of BM and other bodily fluids, and reduced run-off.

### SUMMARY

The present disclosure provides improved three-dimensional multi-layer apertured materials such as nonwoven materials, and absorbent articles comprising the same, having improved absorbency and retention of BM and other bodily fluids and reduced run-off. Absorbent articles may use the three-dimensional multi-layer apertured materials as topsheets, for example. The three-dimensional multi-layer apertured materials may comprise apertures in one or more layers of the multi-layer materials and may create significant void volume for better absorbency, retention, and reduced run-off of BM and other bodily fluids. The apertures may allow BM and other bodily fluids to quickly penetrate into the absorbent articles, while the increased void volumes may allow for better retention of BM or other bodily fluids. Further, the increased void volumes may reduce the spread of BM and other bodily fluids, once captured, thereby providing reduced run-off benefits. The multi-layer apertured materials may also comprise a nested laminate that may result in a further increase in void volume. Additionally, the three-dimensional multi-layer apertured materials of the present disclosure may act to wipe BM or other bodily fluids off of or to wick BM or other bodily fluids away from the skin of a wearer, during wearer movement. Also, the three-dimensional multi-layer apertured materials of the present disclosure may provide high surface areas and contact with the skin to entangle BM or other bodily fluids and at least reduce BM or other bodily fluids from sticking to the skin.

In accordance with an aspect of the present disclosure, an absorbent article is provided comprising: a liquid permeable nonwoven topsheet; a nonwoven second material, wherein the second material may be a separate material from the topsheet; a liquid impermeable backsheet; and an absorbent core positioned at least partially intermediate the second material and the liquid impermeable backsheet. The second material may be positioned intermediate the topsheet and the absorbent core. The topsheet may be nested with the second material to form a nested laminate, wherein the nested laminate comprises a plurality of recesses and a plurality of raised areas, and wherein voids may be defined in the raised areas under the second material. A first aperture may be formed in a substantially central location of at least a majority of the raised areas. The first aperture may extend through both the topsheet and the second material. The recesses may each form a base positioned most distal from the substantially central locations of the raised areas. The second aperture may be formed in at least a majority of the bases of the recesses. The second aperture may extend through both the topsheet and the second material. Areas of the recesses and the raised areas intermediate the first apertures and the second apertures may be free of any apertures.

In accordance with an aspect of the present disclosure, an absorbent article is provided comprising: a liquid permeable nonwoven topsheet; a nonwoven second material, wherein the second material may be a separate material from the topsheet; a liquid impermeable backsheet; and an absorbent core positioned at least partially intermediate the second material and the liquid impermeable backsheet. The second material may be positioned intermediate the liquid permeable topsheet and the absorbent core. The topsheet may comprise a plurality of recesses and a plurality of raised areas. The second material may be generally planar. Portions of the topsheet recesses may be joined to portions of the second material. A first aperture may be formed in a substantially central location of at least a majority of the raised areas. The first aperture may extend through only the topsheet. The recesses may each comprise a base positioned most distal from the substantially central locations of the raised areas. A second aperture may be formed in at least a majority of the bases of the recesses. The second aperture may extend through both the topsheet and the second material. Areas of the recesses and the raised areas intermediate the first apertures and the second apertures may be free of any apertures. A void may be defined intermediate a garment-facing surface of the topsheet and a wearer-facing surface of the second material in the raised areas.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article of the present disclosure that is a sanitary napkin;

FIG. 13 is an example cross-sectional view taken within a front waist region of an absorbent article;

FIG. 14 is an example cross-sectional view taken within a crotch region of an absorbent article;

FIG. 15 is an example cross-sectional view taken within a back waist region of an absorbent article;

FIG. 16 is a detailed plan view of a portion of an example absorbent article, a wearer-facing surface facing the viewer, comprising a multi-layer material of the present disclosure, the material having a plurality of three-dimensional features in the form of raised areas and recesses;

FIG. 17A is a cross-sectional view taken about line 17A-17A of FIG. 16, in which the raised areas comprise a substantially convex outer portion;

FIG. 17B is a cross-sectional view taken about line 17B-17B of FIG. 16;

FIG. 17C is an enlarged view of a raised area or a recess aperture;

FIG. 17D is a view taken along section line 17D-17D in FIG. 17C;

FIG. 18 is a cross-sectional view, similar to FIG. 17A, of an alternative form of an absorbent article comprising a multi-layer material in which the raised areas and recesses comprise a substantially planar outer portion;

FIG. 19 is a detailed plan view of a portion of another example absorbent article, a wearer-facing surface facing the viewer, comprising a multi-layer material of the present disclosure, the material having a plurality of three-dimensional features in the form of raised areas;

FIG. 20 is a cross-sectional view taken about line 20-20 of FIG. 19, in which the raised areas comprise a substantially convex outer portion;

FIG. 21 is a cross-sectional view, similar to FIG. 20, of an alternative form of an absorbent article comprising a multi-layer material in which the raised areas comprise a substantially planar outer portion;

FIG. 22 is a detailed plan view of a portion of a further example absorbent article, a wearer-facing surface facing the viewer, comprising a multi-layer material of the present disclosure, the material having a plurality of three-dimensional features in the form of raised areas and recesses and comprising substantially planar sections between the raised areas and recesses;

FIG. 23 is a cross-sectional view taken about line 23-23 of FIG. 22;

FIGS. 24 and 25 are views of an alternative form of the absorbent article of FIGS. 16 and 17A, respectively, in which the raised areas form more than 50% of the topsheet;

FIGS. 26 and 27 are views of an alternative form of the absorbent article of FIGS. 19 and 20, respectively, in which the raised areas form more than 50% of the topsheet;

FIG. 28 is a plan view of an absorbent article, a wearer-facing surface facing the viewer, comprising a multi-layer material of the present disclosure;

FIG. 29 is a detailed plan view of a portion of a further example absorbent article, a wearer-facing surface facing the viewer, comprising a multi-layer material of the present disclosure, the material having a plurality of planar sections and a plurality of discrete recesses; and

FIG. 30 is a cross-sectional view taken about line 30-30 of FIG. 29.

### DETAILED DESCRIPTION

Various non-limiting forms of the present disclosure will now be described to provide an overall understanding of the principles of the structure, function, manufacture, and use of the three-dimensional materials having apertures disclosed herein. One or more examples of these non-limiting forms are illustrated in the accompanying drawings. Those of ordinary skill in the art will understand that the three-dimensional materials having apertures described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### Definition of Terms

The term “absorbent article” may include disposable articles such as sanitary napkins, panty liners, tampons, interlabial devices, wound dressings, pants, taped diapers, adult incontinence articles, wipes, and the like. At least some of such absorbent articles are intended for the absorption of body liquids, such as menses or blood, vaginal discharges, urine, and feces. Wipes may be used to absorb body liquids, or may be used for other purposes, such as for cleaning surfaces. The nonwoven materials described herein may comprise at least part of other articles such as scouring pads, wet or dry-mop pads (such as SWIFFER® pads), paper towels, toilet tissue, and the like.

The term “aperture”, as used herein, refers to a predetermined and intentional hole that extends completely through a web or structure (that is, a through hole). The apertures may either be formed cleanly through the web so that the material surrounding the aperture lies in the same plane as the web prior to the formation of the aperture (a “two dimensional” aperture), or the holes may be formed such that at least some of the material surrounding the opening is pushed out of the plane of the web. In the latter case, the apertures may resemble a depression with an aperture therein, and may be referred to herein as a “three dimensional” aperture, a subset of apertures. The term “aperture” does not refer to unintentional variances in the nonwoven material, unintentional tears formed during manufacturing, or pores in the nonwoven materials.

The term “disposable” may be used herein to describe absorbent articles and other products which are not intended to be laundered or otherwise restored or reused as an absorbent article or product (i.e., they are intended to be discarded after use and, preferably, to be recycled, composted or otherwise disposed of in an environmentally compatible manner).

The term “joined to” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element. The term “joined to” encompasses configurations in which an element is secured to another element at selected locations, as well as configurations in which an element is completely secured to another element across the entire surface of one of the elements. The term “joined to” includes any known manner in which elements may be secured including, but not limited to mechanical entanglement.

The term “machine direction” or “MD” means the path that material, such as a web, follows through a manufacturing process.

The term “laminate”, as used herein, may refer to an intimate combination of two components, e.g., a topsheet and a second material, wherein the second material may comprise one or more of an acquisition layer, a distribution layer, or another layer, both disposed in a face to face relationship. The topsheet has a first and a second surface. The first surface of the topsheet is facing towards the body of the wearer when the absorbent article is in use. The second material is facing the backsheet when the absorbent article is in use. The topsheet and the second material may undergo a simultaneous and joint mechanical deformation. The laminate may be formed by nesting together the topsheet and the second material, in which three-dimensional features formed in the topsheet coincide with and fit closely together with three-dimensional features of the second material. In a nested laminate, the three-dimensional features may be formed at the same time in both materials.

The term “web” may be used herein to refer to a material whose primary dimension is X-Y, i.e., along its length (or longitudinal direction) and width (or transverse direction). It should be understood that the term “web” is not necessarily limited to single layers or sheets of material. Thus, the web may comprise laminates or combinations of several sheets of the requisite type of materials.

### General Description of an Absorbent Article

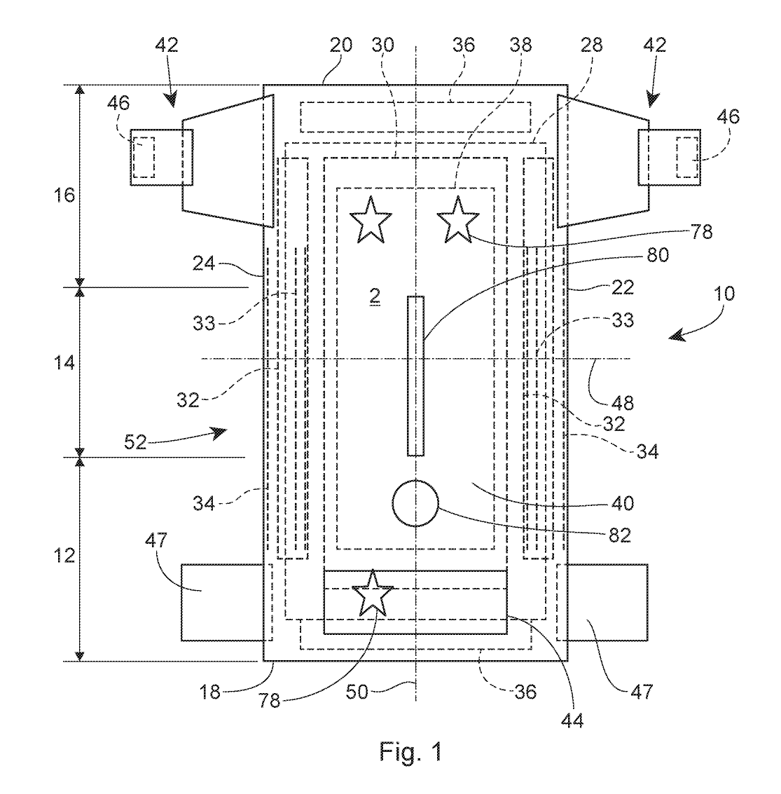

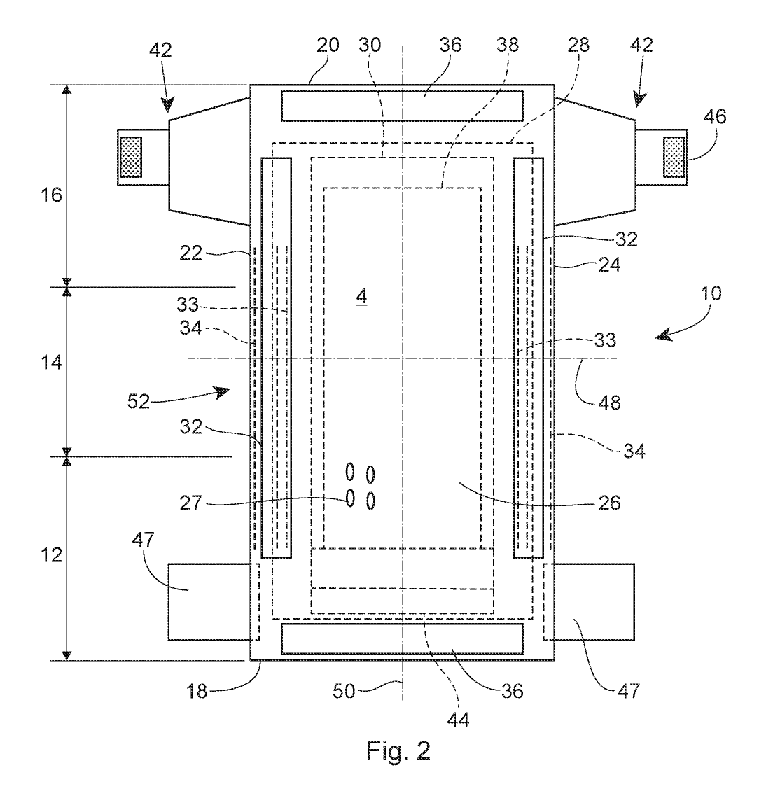

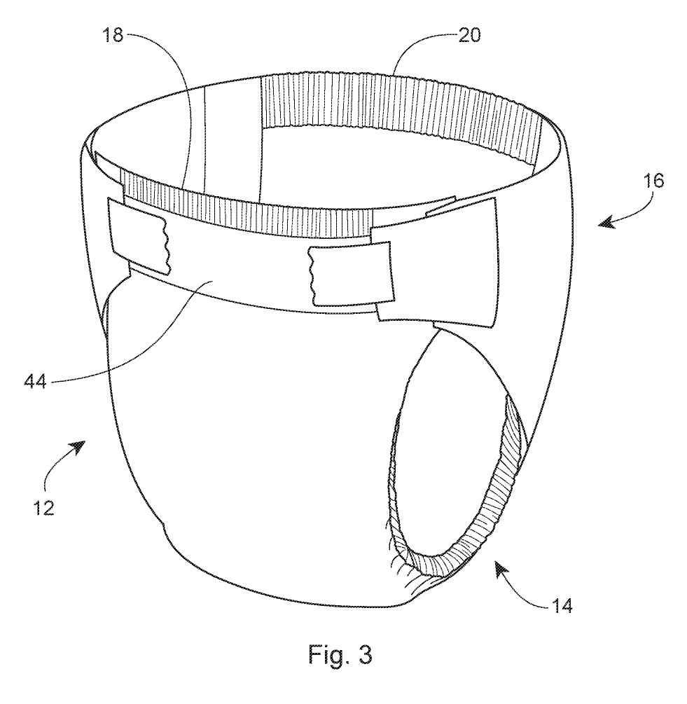

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

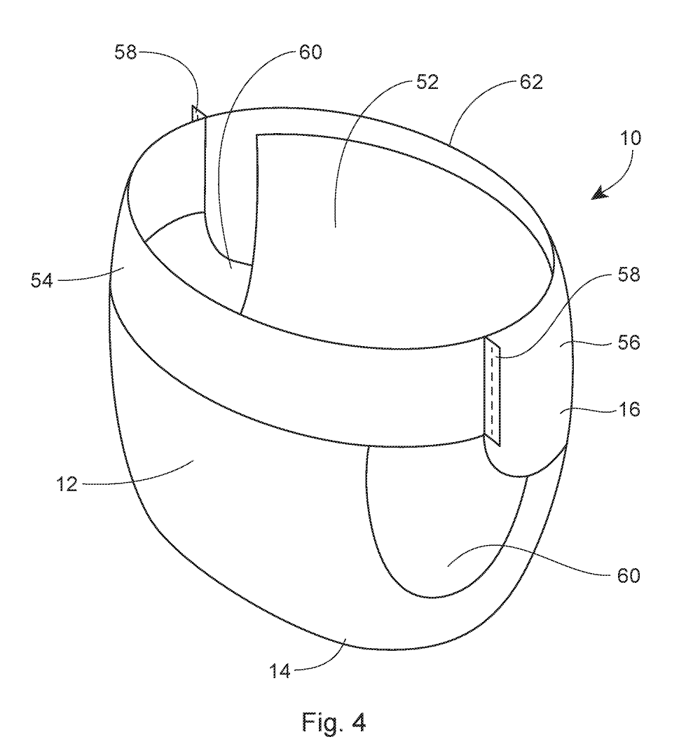

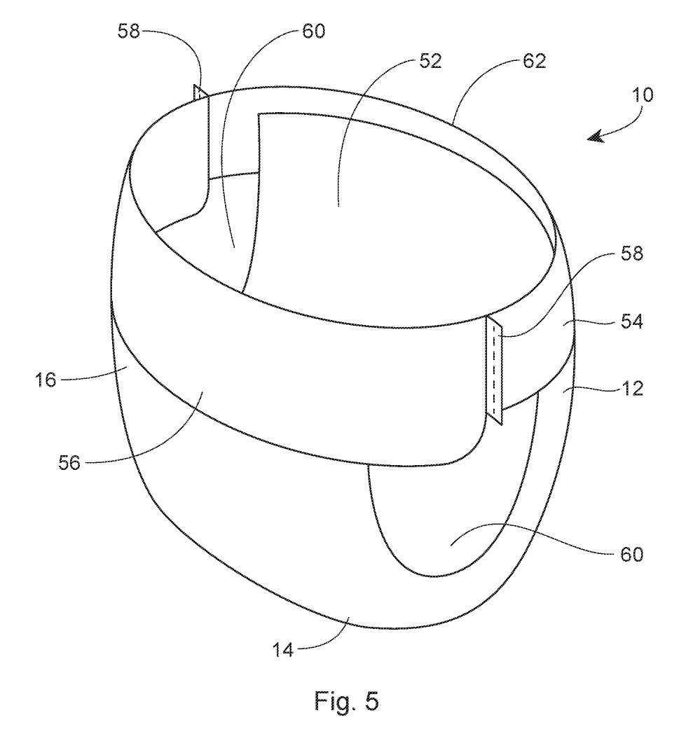

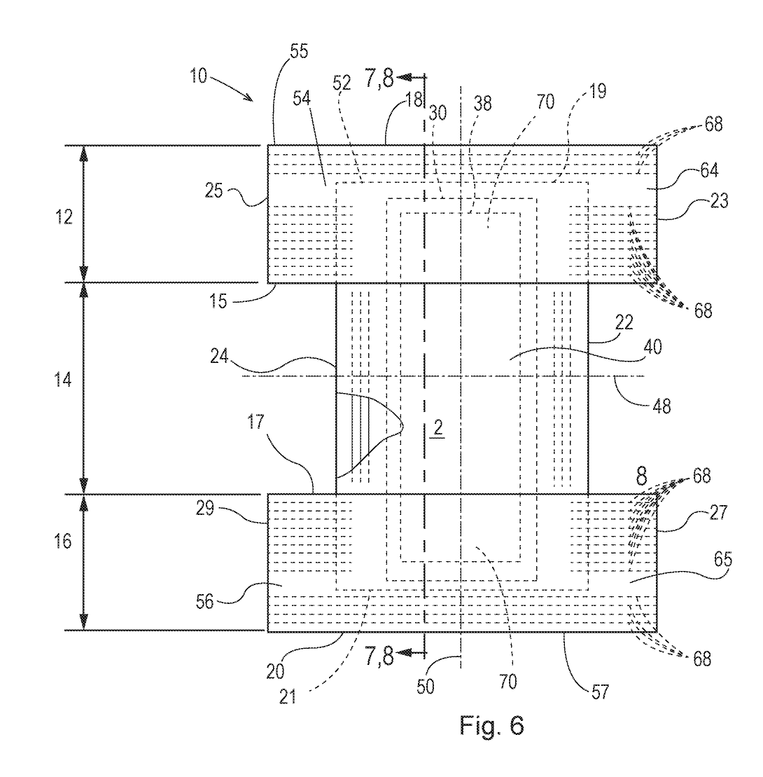

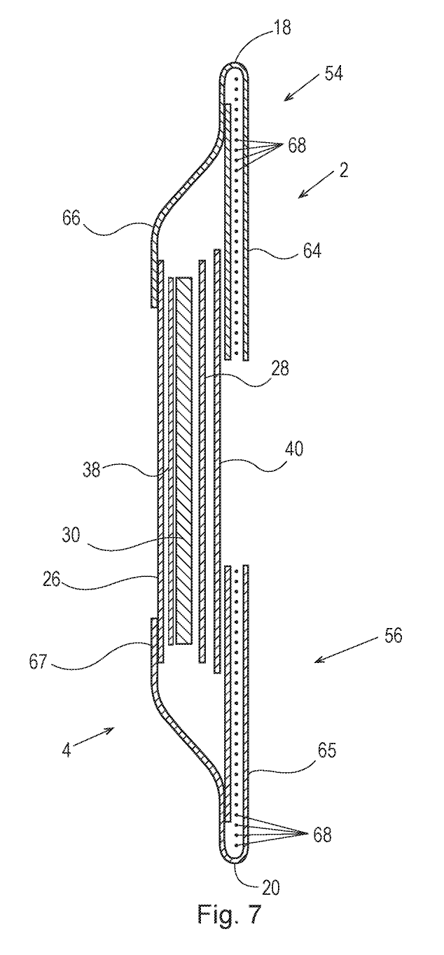

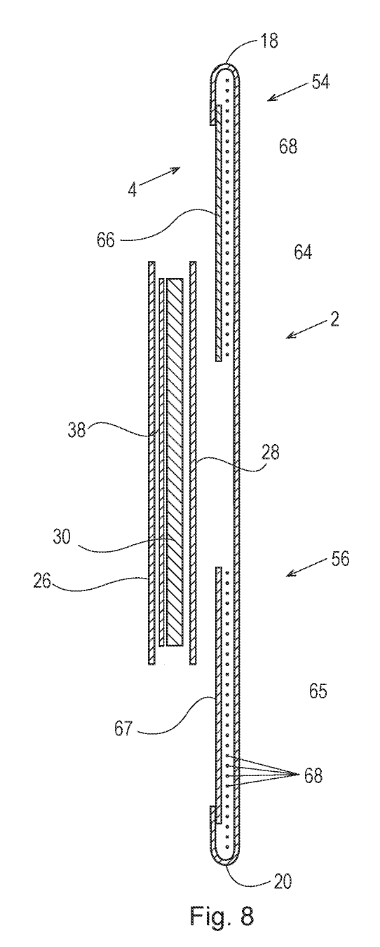

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIG. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIG. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 27), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features.

### Absorbent Core

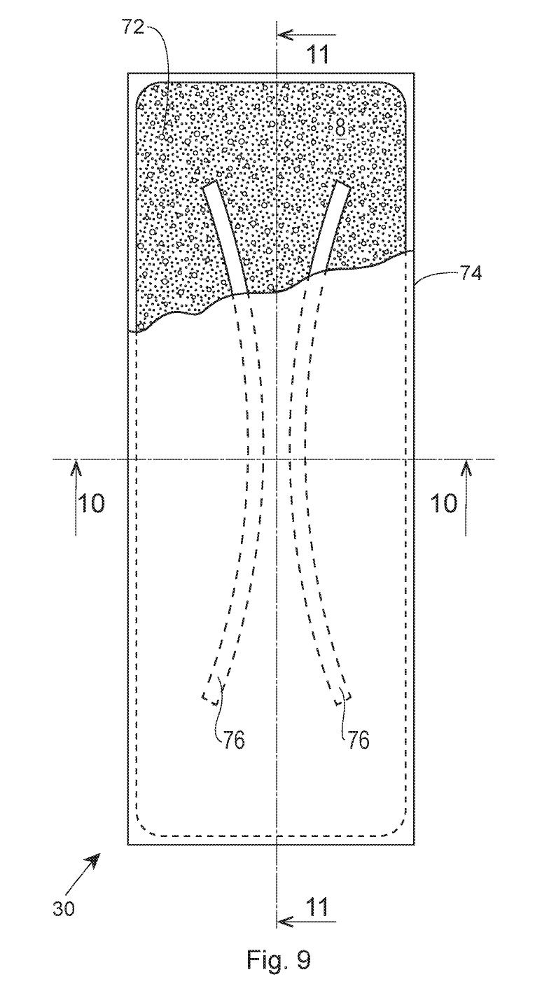

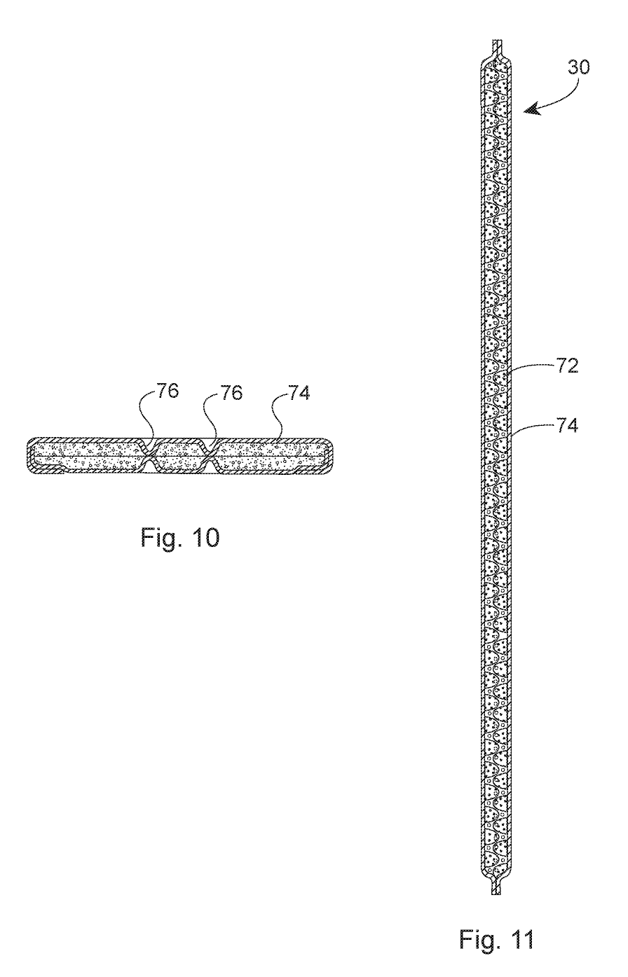

As used herein, the term “absorbent core” 30 refers to the component of the absorbent article 10 having the most absorbent capacity and that comprises an absorbent material. Referring to FIGS. 9-11, in some instances, absorbent material 72 may be positioned within a core bag or a core wrap 74. The absorbent material may be profiled or not profiled, depending on the specific absorbent article. The absorbent core 30 may comprise, consist essentially of, or consist of, a core wrap, absorbent material 72, and glue enclosed within the core wrap. The absorbent material may comprise superabsorbent polymers, a mixture of superabsorbent polymers and air felt, only air felt, and/or a high internal phase emulsion foam. In some instances, the absorbent material may comprise at least 80%, at least 85%, at least 90%, at least 95%, at least 99%, or up to 100% superabsorbent polymers, by weight of the absorbent material. In such instances, the absorbent material may free of air felt, or at least mostly free of air felt. The absorbent core periphery, which may be the periphery of the core wrap, may define any suitable shape, such as rectangular “T,” “Y,” “hour-glass,” or “dog-bone” shaped, for example. An absorbent core periphery having a generally “dog bone” or “hour-glass” shape may taper along its width towards the crotch region 14 of the absorbent article 10. The periphery of the absorbent material within the core bag may also be shaped.

Referring to FIGS. 9-11, the absorbent core 30 may have areas having little or no absorbent material 72, where a wearer-facing surface of the core bag 74 may be joined to a garment-facing surface of the core bag 74. These areas having little or no absorbent material may be referred to as “channels” 76. These channels can embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that providing significant wicking of bodily exudates. These materials may dewater the topsheet 26 and quickly move bodily exudates into the absorbent core 30. The acquisition materials 38 may comprise one or more nonwoven materials, foams, cellulosic materials, cross-linked cellulosic materials, air laid cellulosic nonwoven materials, spunlace materials, or combinations thereof, for example. In some instances, portion of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic stands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

### Arrays

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

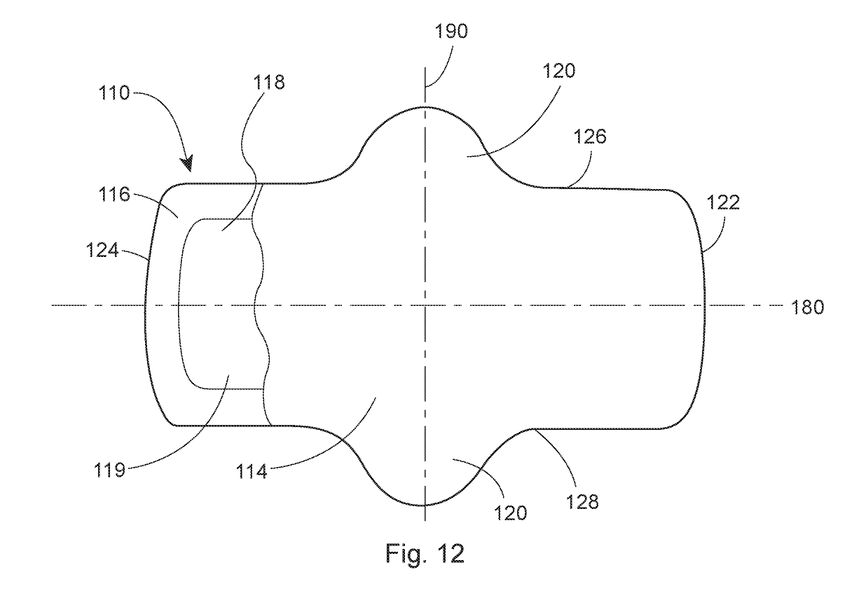

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Examples Cross-Sections of Absorbent Articles

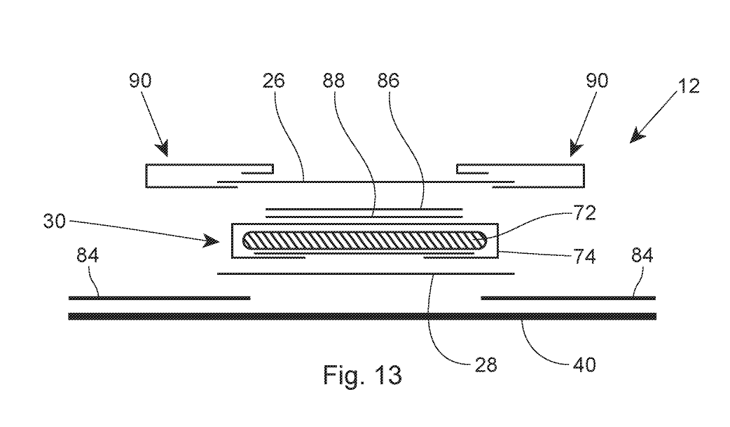

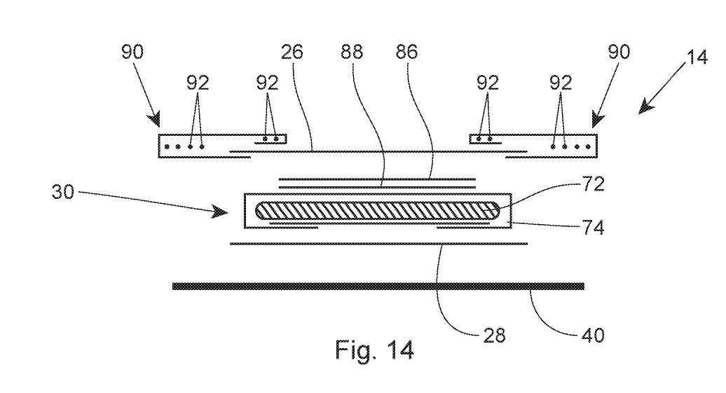

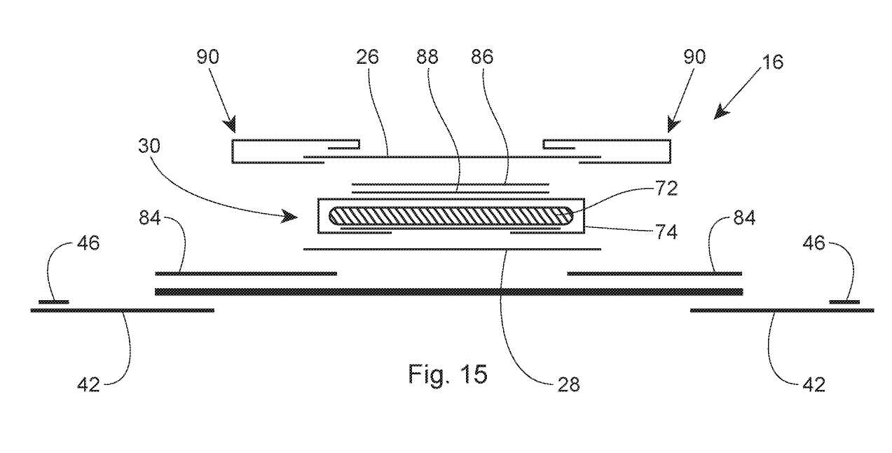

FIGS. 13-15 illustrate example cross-sectional views of absorbent articles within the scope of the present disclosure. FIG. 13 is an example cross-sectional view taken within a front waist region 12 of an absorbent article. FIG. 14 is an example cross-sectional view taken within a crotch region 14 of an absorbent article. FIG. 15 is an example cross-sectional view taken within a back waist region 16 of an absorbent article. In FIGS. 13-15, an outer cover material is element 40, a liquid permeable topsheet is element 26, opacity patches are elements 84, a liquid impermeable backsheet is element 28, an absorbent core is element 30, with the core bag being element 74, an absorbent material is element 72, and a distribution material is element 86. The distribution material 86 may comprise cross-linked cellulosic material and may be optional. An acquisition material is element 88. A liquid permeable topsheet is element 26. Barrier leg cuffs are elements 90. Elastics in the barrier leg cuffs are elements 92. Back ears are elements 42.

Fasteners on the back ears 42 are elements 46. Construction glues and/or bonds between the various layers and/or components have been removed for clarity. Other cross-sectional configurations known to those of skill in the art are also within the scope of the present disclosure.

### General Structure and Properties of a Multi-Layer Material of an Absorbent Article

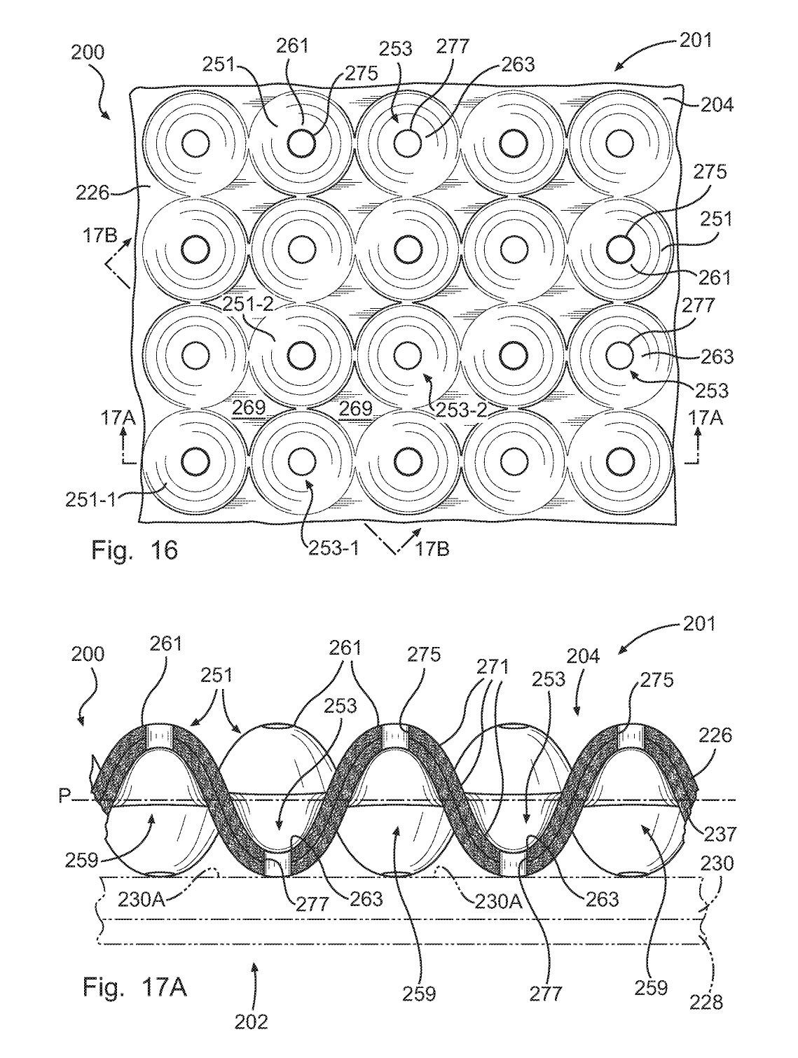

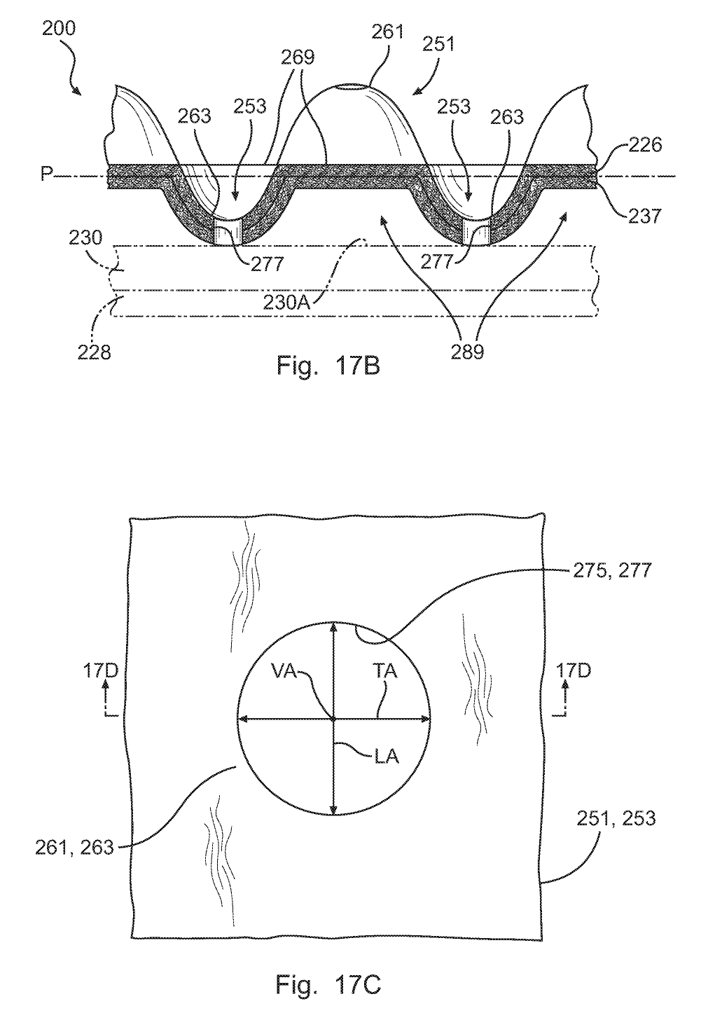

FIG. 16 is a plan view of a portion of an example absorbent article 201 comprising a multi-layer material 200 according to the present disclosure, in which a wearer-facing surface 204 of the absorbent article is facing the viewer. FIGS. 17A and 17B are cross-sectional views of the absorbent article 201 of FIG. 16 taken along view lines 17A-17A and 17B-17B, respectively.

The multi-layer material 200 of FIGS. 16, 17A, and 17B may comprise a liquid permeable topsheet 226 and a second material 237. The absorbent article 201 may comprise the multi-layer material 200, a liquid impermeable backsheet 228, and an absorbent core 230 positioned at least partially intermediate the second material 237 and the backsheet 228, see FIG. 17A. The second material 237 may be positioned intermediate the topsheet 226 and the absorbent core 230 and may define one or more acquisition or distribution material layers or another layer of the topsheet. If the second material 237 discussed herein is another layer of the topsheet, one or more acquisition or distribution layers may also be provided in an absorbent article. The topsheet 226 and the second material 237 together may be used as topsheets, outer cover nonwovens, acquisition layers, topsheet/acquisition layer laminates, topsheet/distribution layer laminates, or various other components of absorbent articles, for example. This description also applies to other example forms discussed herein. Although a particular order of material layers is depicted herein, those of skill in the art will recognize that variances in this order may be possible. Further, a distribution layer may be positioned intermediate the second material 237 and the absorbent core 230. This distribution layer may comprise cross-linked cellulosic fibers, for example.

In some forms of the multi-layer material, such as the multi-layer material 200 of FIG. 16, the second material 237 may be a separate material from the topsheet 226. As noted above, the topsheet 226 and acquisition materials, e.g., the second material 237, may be formed from or may comprise nonwoven materials. The topsheet 226 and the second material 237 may comprise the same materials or different materials, or may comprise the same material with different treatments (e.g., one material may be more hydrophobic or hydrophilic than the other material).

The nonwoven materials of the multi-layer material of the present disclosure may be made of any suitable nonwoven web materials (“precursor materials”). The nonwoven webs may be made from a single layer, or multiple layers (e.g., two or more layers, three or more layers, etc.). If multiple layers are used, they may be comprised of the same type of nonwoven material, or different types of nonwoven materials. In some cases, the precursor materials may be free of any film layers.

The fibers of the nonwoven precursor material(s) may be made of any suitable materials including, but not limited to natural materials, synthetic materials, and combinations thereof. Suitable natural materials may comprise, but are not limited to cellulose, cotton linters, bagasse, wool fibers, silk fibers, etc. Cellulose fibers may be provided in any suitable form, comprising but not limited to individual fibers, fluff pulp, drylap, liner board, etc. Suitable synthetic materials may comprise, but are not limited to nylon, rayon and polymeric materials. Suitable polymeric materials may comprise, but are not limited to: polyethylene (PE), polyester, polyethylene terephthalate (PET), polypropylene (PP), and co-polyester. In some forms, however, the nonwoven precursor materials may be either substantially, or completely free, of one or more of these materials. For example, in some forms, the precursor materials may be substantially free of cellulose, and/or exclude paper materials. In some forms, one or more precursor materials may comprise up to 100% thermoplastic fibers. The fibers in some cases may, therefore, be substantially non-absorbent.

The nonwoven precursor webs may be formed from various suitable processes, such as, for example, air laying processes, wetlaid processes, meltblowing processes, spunbonding processes, and carding processes. The fibers in the webs may then be bonded via spunlacing processes, hydroentangling, calendar bonding, through-air bonding and resin bonding. Some of such individual nonwoven webs may have bond sites where the fibers are bonded together.

The basis weight of nonwoven materials is usually expressed in grams per square meter (gsm). The basis weight of a single layer nonwoven material may range from about 8 gsm to about 100 gsm, depending on the ultimate use of the material. For example, the topsheet 226 of a topsheet/acquisition layer (second material 237) laminate or composite may have a basis weight from about 8 to about 40 gsm, from about 8 to about 30 gsm, or from about 8 to about 20 gsm, for example. An acquisition layer (second material 237) may have a basis weight from about 10 to about 200 gsm, from about 10 to about 150 gsm, or from about 10 to about 80 gsm, for example. Hence, the basis weight of the second material 237 (e.g., a single acquisition layer) may be greater than the basis weight of the topsheet 226. The basis weight of a multi-layer material is the combined basis weight of the constituent layers and any other added components. The basis weight of multi-layer materials of interest herein (e.g., a topsheet 226 and a single acquisition layer defining the second material 237) may range from about 20 gsm to about 250 gsm, depending on the ultimate use of the material. The nonwoven precursor webs may have any suitable density.

The precursor nonwoven webs may have certain desired characteristics. It is typically desirable for the precursor nonwoven web materials to have extensibility to enable the fibers to stretch and/or rearrange into the form of the protrusions (raised areas) and/or recesses. If the nonwoven webs are comprised of two or more layers, it may be desirable for all of the layers to be as extensible as possible. It may also be desirable for the precursor nonwoven webs to be capable of undergoing plastic deformation to ensure that the structure of the deformations is “set” in place so that the nonwoven web will not tend to recover or return to its prior configuration.

When the nonwoven web comprises two or more layers, the different layers may have the same properties, or any suitable differences in properties relative to each other. One of the layers may serve as the topsheet 226, and one or more other layers may define the second material 237 and may comprise one or more acquisition layers. The acquisition layer(s) receives liquids that pass through the topsheet 226 and acquires and/or distributes them, for example, to the underlying absorbent core 230. In some forms, the topsheet 226 may comprise a hydrophobic material, and the second material 237 may comprise a hydrophilic material. In some forms, the topsheet may be more hydrophobic than the second material 237. In other forms, the topsheet 226 may be less hydrophilic than the second material 237, which may lead to better dewatering of the topsheet 226. Hence, a hydrophilicity gradient may be defined such that it extends from a wearer-facing surface of the topsheet 226 to a garment-facing surface of the second material 237 and increases from the wearer-facing surface of the topsheet 226 to the garment-facing surface of the second material 237. In other aspects, the topsheet 226 may be more hydrophilic than the second material 237.

Two or more layered nonwoven webs may be combined together in any suitable manner. In some cases, the layers may be unbonded to each other and held together autogenously (that is, by virtue of the formation of deformations therein). For example, two precursor webs from which the topsheet 226 and second material 237 are formed contribute fibers to deformations in a “nested” relationship that joins the two precursor webs together, forming a multi-layer material without the use or need for adhesives or thermal bonding between the layers. In other aspects, the layers may be joined together by other mechanisms. If desired, an adhesive between the layers, ultrasonic bonding, chemical bonding, resin or powder bonding, thermal bonding, or bonding at discrete sites using a combination of heat and pressure may be selectively utilized to bond certain regions or all of the precursor webs. In addition, the multiple layers may be bonded during processing, for example, by carding one layer of nonwoven onto a spunbond nonwoven and thermal point bonding the combined layers. In some cases, certain types of bonding between layers may be excluded. For example, the layers of the present example materials disclosed herein may be non-hydroentangled together.

In the example depicted in FIGS. 17A and 17B, the topsheet 226 is nested with the second material 237 to form a nested laminate comprising a plurality of three-dimensional elements in the form of raised areas 251 and recesses 253. Although each is depicted as one layer, the topsheet 226 and/or second material 237 may each comprise two or more layers of material. Each of the raised areas 251 may comprise curved sidewalls 271 extending upward in a direction away from the absorbent core 230 and forming a substantially convex outer portion. The raised areas 251 may be dome shaped (i.e., shaped similar to a partial sphere) as shown in FIGS. 17A and 17B, and in other aspects, the raised areas 251 may have a parabolic shape in cross section, a substantially conical or frustoconical shape, or any other curvilinear cross section or configuration. Each of the recesses 253 may comprise curved sidewalls 271 extending downward in a direction toward the absorbent core 230 (e.g., toward a garment-facing surface 202 of the absorbent article) and forming a substantially concave outer portion, i.e., concave inwardly from the wearer facing surface of the nested laminate. The recesses 253 may comprise a dome shape (i.e., shaped similar to a partial sphere and inverted relative to the raised areas 251) as shown in FIGS. 17A and 17B, and in other aspects, the recesses 253 may have a parabolic shape in cross section, a substantially conical or frustoconical shape, or any other curvilinear cross section or configuration. In further aspects, one or more sections of the sidewalls 271 defining the raised areas 251 and/or the recesses 253 may be substantially linear or have any other suitable shape, such as an S-like shape, for example.

As shown in FIGS. 16 and 17B, substantially planar sections 269 of the multi-layer material 200 may be located between pairs of adjacent raised areas 251, i.e., diagonally positioned raised areas 251-1 and 251-2, and pairs of adjacent recesses 253, i.e., diagonally positioned recesses 253-1 and 253-2. The substantially planar sections 269 may be positioned in a plane P located generally between the raised areas 251 and the recesses 253, see FIG. 17B. From FIGS. 17A and 17B, it may be seen that the raised areas 251 and recesses 253 extend above and below the plane P, while the planar sections 269 may be generally located within or substantially parallel to the plane P. A space 289 may be defined between each planar section 269 and an adjacent layer of material. For example, as shown in FIG. 17B, the spaces 289 may be defined intermediate a garment-facing surface of the second material 237 of the planar sections 269 and a wearer-facing surface of the absorbent core 230 or core bag. The absorbent material within the absorbent core 230 may be contained within a core bag, as discussed above.

The three-dimensional features, i.e., the raised areas 251 and recesses 253, may be disposed in any suitable density across the surface of the nested laminate. The features may, for example, be present in a density of: from about 5 to about 100 features; alternatively, from about 10 to about 50 features; alternatively, from about 20 to about 40 features, in an area of 10 cm².

An upper portion of each raised area 251 may define a substantially central location 261 comprising a highest point or a highest section including the highest point of the raised area 251, i.e., furthest from the plane P. The highest point may also be referred to as a crest or apex. The raised area upper portion defines the substantially convex outer portion noted above. Each recess 253 may comprise a trough or base defining a substantially central location 263 comprising a lowest point or a lowest section including the lowest point of the recess 253, i.e., furthest from the plane P toward the garment-facing surface 202 of the absorbent article. Thus, the central locations 261 of the raised areas 251 may each comprise the point or section that is positioned most distal from the central locations or bases 263 of the recesses 253, and the bases 263 of the recesses 253 may each comprise the point or section that is positioned most distal from the central locations 261, e.g., the central location highest points, of the raised areas 251. Reference may also be made to the plane P, in which the central location 261 of each raised area 251 comprises the raised area point or section that is positioned most distal relative to the plane P, and the central location 263 of each recess 253 comprises the recess point or section that is positioned most distal relative to the plane P. Process tolerances should be taken into account for the substantially central locations 261, 263.

A void 259 may be defined between each raised area 251 and a substantially planar area 230A under the second material 237, which planar area 230A may be defined in the FIG. 17A example by the wearer-facing surface of a substantially planar region of the absorbent core 230. For example, the voids 259 may be defined intermediate a garment-facing surface of the second material 237 of the raised areas 251 and a wearer-facing surface of an adjacent layer of material such as the wearer-facing surface of the substantially planar region of the absorbent core 230, as shown in the example illustrated in FIGS. 17A and 17B. The term “generally planar” is not meant to imply any particular flatness, smoothness, or dimensionality. Thus, the substantially planar region of the absorbent core 230 may comprise other features that provide the absorbent core 230 with a topography. Such other features may comprise, but are not limited to small projections, raised network regions, and other types of features. Thus, the substantially planar region of the absorbent core 230 is generally planar when considered relative to the raised areas 251 and recesses 253. These voids 259 provide void volume for BM or other bodily fluid retention so that such BM or other bodily fluids may be absorbed into the absorbent core 230 positioned under the nested laminate or may be at least partially dewatered by the absorbent core 230.

In some particular aspects in which the topsheet 226 and the second material 237 comprise a nested laminate, the topsheet 226 may be nested with or joined to the second material 237 in the raised areas 251 and the recesses 253. In some examples, such as the FIG. 17A example, the nested laminate comprising the topsheet 226 and the second material 237 may be joined to the next layer of material in the direction of the garment-facing surface 202 of the material 200, e.g., the absorbent core 230, only at the bases of the recesses 253.

A width or circumference of each of the raised areas 251 and recesses 253 may be greatest at a point nearest the plane P. For example, in the examples shown in FIGS. 16 and 17A, the sidewalls 271 defining each raised area 251 may curve substantially continuously, along at least a section of the raised area 251, toward the substantially central location 261 of the raised area 251, and the sidewalls 271 defining each recess 253 may curve substantially continuously, along at least a section of the recess 253, toward the central location 263 of the recess 253. In other aspects in which one or more sections of the sidewalls 271 are substantially linear, the sidewalls 271 may slope toward the respective central locations 261, 263 of the raised areas 251 and the recesses 253. In further aspects in which the sidewalls 271 are substantially linear, the sidewalls 271 may extend outwardly substantially perpendicular to the plane P, and the width or circumference of each raised area 251 and the recess 253 may remain substantially the same along at least a portion of the raised area 251 and the recess 253 from the point nearest the plane P toward the respective central location 261, 263.

A first aperture 275 may be formed in the substantially central location 261, e.g., at the central location highest point, of each of at least a majority of the raised areas 251, and a second aperture 277 may be formed in the substantially central location or base 263, e.g., at the central location lowest point, of each of at least a majority of the recesses 253, as seen in FIG. 16. In the example shown in FIG. 17A, the apertures 275, 277 extend through both the topsheet 226 and the second material 237. The first apertures 275 formed in the raised areas 251 may be in fluid communication with the voids 259.

By providing apertures 275, 277 through both the topsheet 226 and the second material 237, BM and bodily fluids may be better absorbed and, in the context of an absorbent article, wicked toward the absorbent core 230. BM or other bodily fluids may be able to bypass some of the resistance to acquisition of the topsheet 226 and the acquisition layer(s) (e.g., the second material 237), thereby reducing BM, or other bodily fluid, spreading (i.e., run-off) (especially when the BM, or other bodily fluids are within the voids 259). The apertures 275, 277 also allow the topsheet 226 to acquire urine better while being less hydrophilic or hydrophobic than typical topsheets, thereby leading to better dryness, especially with relatively large aperture dimensions (e.g., greater than 0.75 mm in width and/or length, greater than 1.0 mm in width and/or length, greater than 1.5 mm in width and/or length, or greater than 2.0 mm in width and/or length, for example). This dryer, wearer-facing surface 204 may also lead to reduced skin marking or red marking. A width of the recesses 253, as measured, for example, at the plane P in FIG. 17A, may be less than 3 mm, which may also help to reduce skin marking.

Some current two-dimensional apertured topsheets are somewhat effective at allowing BM to pass through the topsheet into the layers below. These two-dimensional apertured topsheets, however, provide very little void volume under the topsheets in that the generally planar topsheets are in a facing relationship with the generally planar layer below (typically an acquisition layer). Thus, the BM or other bodily fluid acquisition of these two-dimensional apertured topsheets has limitations. The three-dimensional nonwoven materials of the present disclosure having apertures provide an improvement in BM or other bodily fluid acquisition, while also providing reduced skin marking and improved softness.

The raised areas 251 and recesses 253 may each comprise one respective aperture 275, 277, as shown in FIGS. 16 and 17A. The apertures 275, 277 may comprise any suitable shape, including cylindrical, ovate, diamond-shaped, etc., when viewed from the wearer-facing surface of the topsheet 226. In some aspects, a major axis, e.g., a longitudinal axis LA, defining a length of each aperture 275, 277 may be less than 4 mm, see FIG. 17C. In some particular aspects, the longitudinal axis LA of each aperture 275, 277 may be less than 3 mm. Depending on the shape of the aperture 275, 277, an axis TA defining a width of each aperture 275, 277 and being substantially transverse to the longitudinal axis LA may be less than or equal to the longitudinal axis LA. For apertures 275, 277 having a substantially cylindrical shape, the longitudinal and transverse axes LA and TA may be generally equal and define a cylinder diameter. In the depicted examples, a thickness T of the apertures 275, 277 parallel to a vertical axis VA, wherein the vertical axis VA is perpendicular to the longitudinal and transverse axes LA and TA, may be substantially uniform through the entire thickness of the topsheet 226 and second material 237 when viewed in cross-section, see FIG. 17D.

In some aspects, the apertures 275, 277 may all be of a similar size and/or shape, and in other aspects, the apertures 275, 277 may be of a different size and/or shape. In some examples, the apertures 275 in the raised areas 251 may be less than 2 mm, and in other examples, the apertures 275 in the raised areas 251 may be smaller than the apertures 277 in the recesses 253, both of which may help to reduce skin marking.

Although the structures immediately surrounding the apertures 275, 277 in FIG. 17D are illustrated as planar, this is merely to illustrate the concept of aperture thickness. In other aspects, the apertures 275, 277 may form a substantially conical or frustoconical shape, e.g., a shape that narrows at one end toward either the garment-facing surface or the wearer-facing surface of the nested laminate. The topsheet 226 may have a thickness T_{TS} of from about 50 microns to about 2 mm and the second material 237 may have a thickness T_{SM} from about 100 microns to about 5 mm, see FIG. 17D. In further aspects, the raised areas 251 and/or recesses 253 may each comprise two or more apertures. In yet further aspects, the raised areas 251 may be continuous.

Any suitable processes for forming the apertures 275, 277 may be utilized. For example, the apertures 275, 277 in the materials of the present disclosure may be formed by hydroforming carded webs, laser cutting, punching with a patterned roll, die cutting, using hot pin methods, overbonding and ring rolling aperturing, as disclosed in U.S. Patent Application Publication No. US 2016/0136014 and U.S. Pat. No. 5,628,097, or other suitable methods. The materials could also be apertured by hand, using a pin punch, for example. Additional example aperturing processes may be used such as described in U.S. Pat. Nos. 9,023,261, 8,158,043, 8,241,543, and 8,679,391.

Any suitable processes for forming three-dimensional features may be used. For example, three-dimensional features may be formed by hydroforming or airlaying on a patterned belt or drum, male/female embossing, pin to flat embossing. Additional example processes for forming the three-dimensional features are disclosed in U.S. Pat. Appl. Publ. Nos. 2015/0250662, 2016/0074250 and in U.S. Pat. No. 7,682,686. The three-dimensional features may also be formed by hand.

The areas or portions of the raised areas 251 and the recesses 253 intermediate the apertures 275, 277 may be free of any additional aperture(s). For example, as seen in FIG. 17A, the sidewalls 271 of the raised areas 251 and the recesses 253 extending between the apertures 275, 277 may be continuous and contain no additional apertures. It is believed that by forming the raised areas 251 and the recesses 253 without apertures in the sidewalls 271, the strength of the raised areas 251 and recesses 253 may be enhanced so as to reduce the likelihood that the raised areas 251 and/or recesses 253 may collapse under in-bag compression or compression caused by a wearer.

Some textured topsheets having protrusions extending toward the skin of a wearer and recesses extending toward an absorbent core have limitations regarding handling body fluids, both urine and viscous body fluids such as runny BM. By providing apertures 275, 277 in a majority of the raised areas 251 and recesses 253, enhanced flow occurs of viscous fluids, such as runny BM towards the absorbent core 230 and away from the wearer-facing surface of the topsheet 226 and the skin of a wearer. The first apertures 275 on the raised areas 251 allow the viscous fluids to access more easily the volume of the voids 259 below the raised areas 251. The second apertures 277 in the recesses 253 reduce the likelihood that viscous fluids may pool in those recesses 253 instead of being absorbed by the absorbent core 230 beneath the nested laminate. As noted above, in some examples, a hydrophilicity gradient may be defined such that it extends from the wearer-facing surface of the topsheet 226 to the garment-facing surface of the second material 237 and increases from the wearer-facing surface of the topsheet 226 to the garment-facing surface of the second material 237. In these examples, quick absorption of urine or menses occurs through the apertures 275, 277 in the raised areas 251 and the recesses 253, while leaving a relatively dry topsheet 226, being drained from urine or menses due to the hydrophilicity gradient.

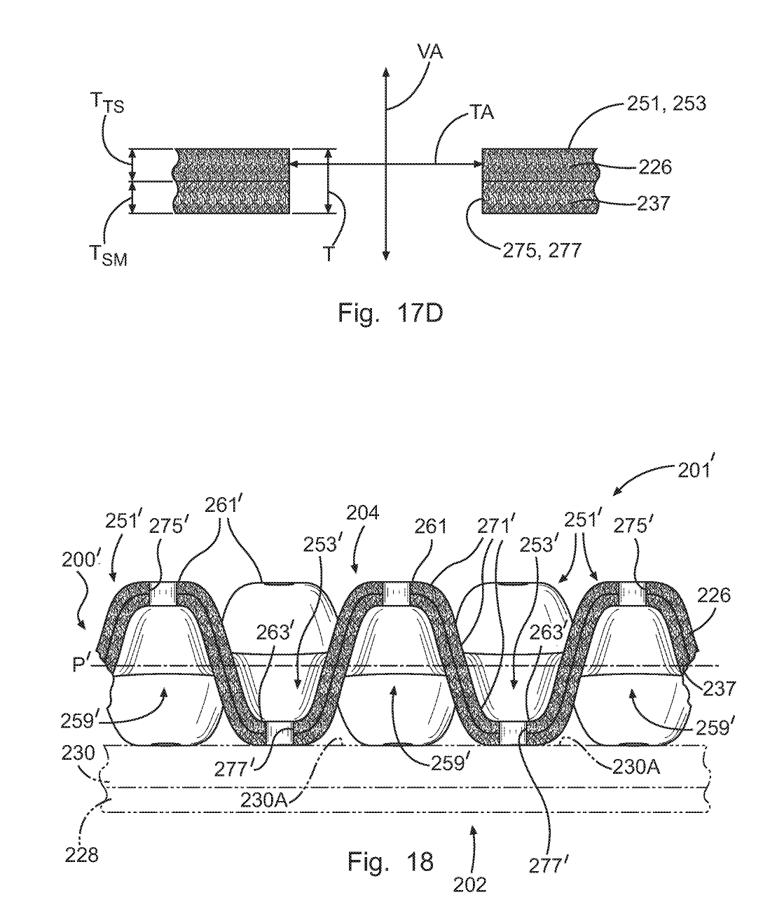

FIG. 18 is a cross-sectional view of another example absorbent article 201′ comprising a multi-layer material 200′ according to the present disclosure. Unless otherwise noted, the material and configuration of the absorbent article illustrated in FIG. 18 may be substantially similar to the absorbent article depicted in FIGS. 16 and 17A-17D, and like reference numerals identify like elements. In FIG. 18, the topsheet 226 may be nested with the second material 237 to form a nested laminate comprising a plurality of three-dimensional features in the form of raised areas 251′ and recesses 253′.

Each raised area 251′ may comprise curved sidewalls 271′ and an upper portion defined by a substantially planar outer portion 261′, in which the sidewalls 271′ project substantially outwardly from a plane P′ located between the raised areas 251′ and the recesses 253′ and in a direction away from the absorbent core 230 to the planar outer portion 261′. In other aspects, the sidewalls 271′ of the raised areas 251′ may be substantially linear. The planar outer portion 261′ on each raised area 251′ may comprise a substantially central location comprising an upper center point or an upper center section including the upper center point. Substantially any point on the raised area planar outer portion 261′ may define a crest or apex, which may comprise a highest point on the raised area 251′. Each recess 253′ may comprise curved sidewalls and a base defined by a substantially planar portion 263′, in which the sidewalls 271′ project toward the absorbent core 230 and inwardly from the plane P′ to the planar portion 263′. In other aspects, the sidewalls 271′ of the recesses 253′ may be linear or S-shaped. The substantially planar portion 263′ of each recess 253′ may comprise a substantially central location comprising a lower center point or a lower center section including the lower center point. Substantially any point on the recess planar portion or base 263′ may comprise a lowest point of the recess 253′.

A void 259′ may be defined in the raised areas 251′ under the second material 237. For example, the voids 259′ may be defined by a garment-facing surface of the second material 237 of the raised areas 251′ and a wearer-facing surface of the adjacent layer of material such as a substantially planar area 230A of the absorbent core 230. These voids 259′ may provide void volume for BM or other bodily fluid retention so that such BM or other bodily fluids may be absorbed into the absorbent core 230 positioned under the nested laminate or may be at least partially dewatered by the absorbent core 230.

Similar to the example depicted in FIG. 17A, a width or circumference of each of the raised areas 251′ and recesses 253′ of FIG. 18 may be greatest at a point nearest the plane P′.

A first aperture 275′ may be formed in the central location of the planar outer portion 261′, e.g., at the upper center point, of each of at least a majority of the raised areas 251′, and a second aperture 277′ may be formed in the central location of the planar portion or base 263′, e.g., at the lower center point, of each of at least a majority of the recesses 253′. The apertures 275′, 277′ may extend through both the topsheet 226 and the second material 237. The apertures 275′ and 277′ may be shaped and sized in the same manner as described above with regards to the apertures 275 and 277 of the example illustrated in FIGS. 16 and 17A-17D.

The areas or portions of the raised areas 251′ and the recesses 253′ intermediate the apertures 275′, 277′ may be free of any additional aperture(s). For example, as seen in FIG. 18, the sidewalls of the raised areas 251′ and the recesses 253′ extending between the apertures 275′, 277′ may be continuous and contain no additional apertures. The benefits of the multi-layer material 200′ in the example of FIG. 18 are substantially the same as those described above with respect to the multi-layer material 200 of the example of FIGS. 16 and 17A-17D. In further aspects, the raised areas 251′ and/or recesses 253′ may each comprise two or more apertures. In yet further aspects, the raised areas 251′ may be continuous, i.e., free of apertures.

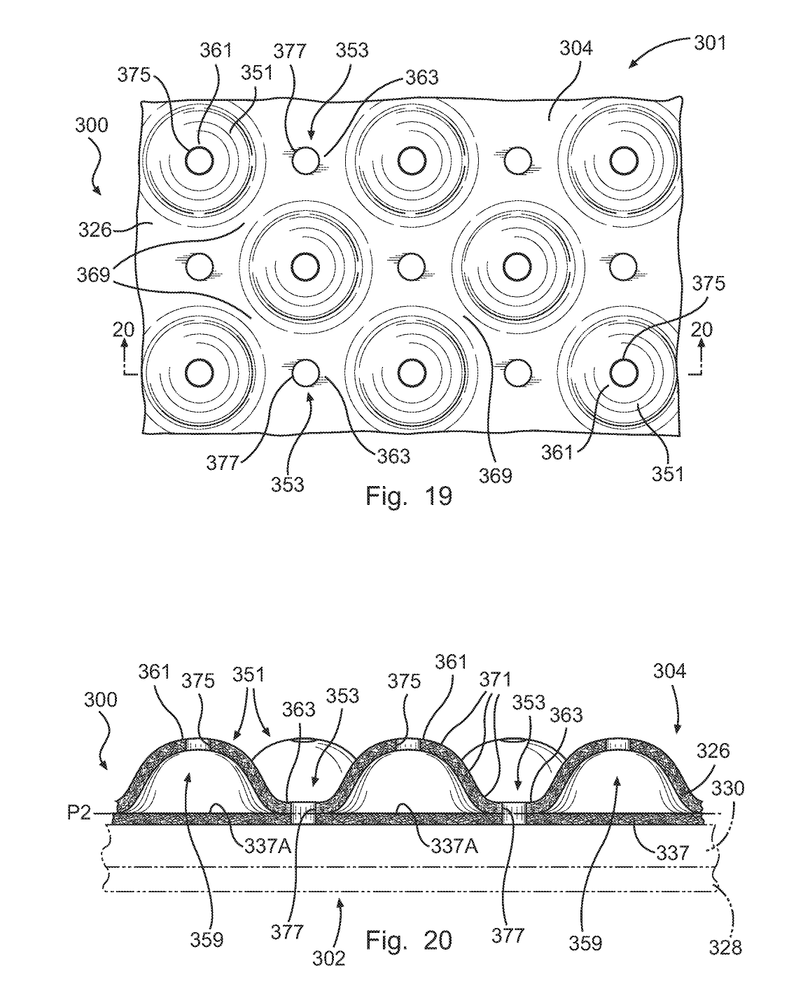

FIG. 19 is a plan view of a portion of another example absorbent article 301 comprising a multi-layer material 300 according to the present disclosure, in which a wearer-facing surface 304 of the absorbent article is facing the viewer. FIG. 20 is a cross-sectional view of the absorbent article of FIG. 19 taken along line 20-20. The multi-layer material 300 may comprise a liquid permeable topsheet 326 and a second material 337 that may be a separate material from the topsheet 326. The absorbent article 301 may comprise the multi-layer material 300, a liquid impermeable backsheet 328, and an absorbent core 330 positioned at least partially intermediate the second material 337 and the backsheet 328. The second material 337 may be positioned intermediate the topsheet 326 and the absorbent core 330 and may comprise one or more acquisition or distribution material layers or merely another layer of the topsheet. The multi-layer material 300 may also be used as an outer cover material, or other absorbent article components, as mentioned above. The topsheet 326 and second material 337 may be formed from the same nonwoven web materials used to form the topsheet 226 and second material 237 of the example of FIGS. 16 and 17A-17D described above. Further, a distribution layer may be positioned intermediate the second material 337 and the absorbent core 330. This distribution layer may comprise cross-linked fibers, for example.

As shown in FIG. 20, the second material 337 may comprise a generally planar material defining a plane P2 along its wearer-facing surface, and the topsheet 326 may comprise a plurality of three-dimensional features in the form of raised areas 351 and recesses 353. Each of the raised areas 351 may comprise curved sidewalls 371 extending upward in a direction away from the absorbent core 330 and the plane P2 and forming a substantially convex outer portion. The raised areas 351 may be dome shaped (i.e., shaped similar to a partial sphere) as shown in FIG. 20, and in other aspects, the raised areas 351 may have a parabolic shape in cross section, a substantially conical or frustoconical shape, or any other curvilinear cross section or configuration. In further aspects, one or more sections of the sidewalls 371 defining the raised areas 351 may be substantially linear. The recesses 353 are adjacent to and located between the raised areas 351 and share topsheet sidewalls 371 with adjacent raised areas 351. The recesses 353 extend in a direction toward the absorbent core 330 and the plane P2 and form a substantially concave outer portion. Depending upon the size of the raised areas 351 and the spacing between the raised areas 351, the recesses 353 may have substantially planar central locations or bases 363. Also, the topsheet 326 may have substantially planar sections 369 located between the raised areas 351 and the recesses 353, see FIG. 19. Both the raised areas 351 and the recesses 353 are located side by side above the plane P2 of the second material 337.

In some aspects, a substantially planar web of topsheet material may be deformed at predefined locations in a direction away from an initial plane of the web of topsheet material to create the raised areas 351 and recesses 353. In contrast to the example of FIGS. 16 and 17A-17D, where the recesses 253 may be deformed into or below the initial plane of the topsheet/second material webs, the recesses 353 in the example of FIGS. 19 and 20 are not deformed into or below the initial plane of the web of topsheet material. However, it is contemplated that the topsheet 326 of FIGS. 19 and 20 could be formed such that the raised areas 351 are deformed into the web of topsheet material to extend above the initial plane of the web of topsheet material and the recesses 353 are deformed into the web of topsheet material to extend below the initial plane of the web of topsheet material.

The three-dimensional features, i.e., the raised areas 351 and recesses 353, may be disposed in any suitable density across the surface of the nested laminate. The features may, for example, be present in a density of: from about 5 to about 100 features; alternatively, from about 10 to about 50 features; alternatively, from about 20 to about 40 features, in an area of 10 cm².

An upper portion of each raised area 351 may define a substantially central location 361 comprising a highest point or a highest section including the highest point of the raised area 351, i.e. furthest from the plane P2. The highest point may also be referred to as a crest or apex. Each recess 353 may comprise a trough or base defining a substantially central location 363 comprising a lowest point or lowest section including the lowest point of the recess 353. Thus, the central locations 361 of the raised areas 351 may each comprise the point or section that is positioned most distal from the central locations or bases 363 of the recesses 353 and from the plane P2, and the bases 363 of the recess 353 may each comprise the point or section that is positioned most distal from the central locations 361 of the raised areas 351.

A void 359 may be defined in the raised areas 351 intermediate a garment-facing surface of the topsheet 326 and a substantially planar area 337A defined by a wearer-facing surface of the generally planar second material 337. The voids 359 may provide generally the same benefits described above with respect to the voids 259 of the example of FIGS. 16 and 17A-17D. The topsheet 326 may be joined to the second material 337 in the recesses 353, e.g., at the bases 363, such as by any of the methods/mechanisms discussed above for joining nonwoven layers together. In some aspects, the topsheet 326 may also be joined to the second material 337 at the generally planar sections 369.

A width or circumference of each raised area 351 may be greatest at a point nearest the plane P2. For example, in the example shown in FIG. 20, the sidewalls 371 defining each raised area 351 may curve substantially continuously, along at least a section of the raised area 351, toward the substantially central location 361 of the raised area 351. In other aspects in which one or more sections of the sidewalls 371 are substantially linear, the sidewalls 371 may slope toward the substantially central location 361 of the raised area 351. In further aspects in which the sidewalls 371 are substantially linear, the sidewalls 371 may extend outwardly substantially perpendicular to the plane P2, and the width or circumference of the raised areas 351 may remain substantially the same along at least a portion of the raised area 351 from the point nearest the plane P2 toward the substantially central location 361.

With reference to FIGS. 19 and 20, a first aperture 375 may be formed in the substantially central location 361, e.g., the highest point, of each of at least a majority of the raised areas 351, and a second aperture 377 may be formed in the substantially central location or base 363, e.g., the lowest point, of each of at least a majority of the recesses 353. As shown in FIG. 20, the first apertures 375 formed in the raised areas 351 extend only through the topsheet 326, while the second apertures 377 formed in the recesses 353 extend through both the topsheet 326 and the second material 337. The first apertures 375 formed in the raised areas 351 may be in fluid communication with the voids 359. The apertures 375 and 377 may be shaped and sized in the same manner as described above with regards to the apertures 275, 277 in the example of FIGS. 16 and 17A-17D.

The raised areas 351 and recesses 353 may each comprise one respective aperture 375, 377, and the areas or portions of the raised areas 351 and the recesses 353 intermediate the apertures 375, 377 may be free of any additional aperture(s). For example, as seen in FIG. 20, the sidewalls 371 of the raised areas 351 and the recesses 353 extending between the apertures 375, 377 are continuous and contain no additional apertures. The benefits of the multi-layer material 300 of the example of FIGS. 19 and 20 are substantially the same as those described above with respect to the multi-layer material 200 of the example of FIGS. 16 and 17A-17D. In further aspects, the raised areas 351 and/or recesses 353 may each comprise two or more apertures. In yet further aspects, the raised areas 351 may be continuous, i.e., free of apertures.

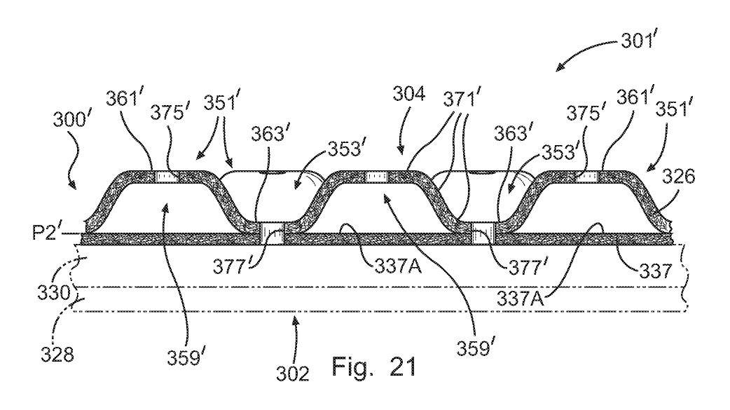

FIG. 21 is a cross-sectional view of another example absorbent article 301′ comprising a multi-layer material 300′ according to the present disclosure. Unless otherwise noted, the material and configuration of the absorbent article illustrated in FIG. 21 may be substantially similar to the absorbent article depicted in FIGS. 19 and 20, and like reference numerals identify like elements.

In FIG. 21, the second material 337 may comprise a generally planar material defining a plane P2′ along its wearer-facing surface, and the topsheet 326 may comprise a plurality of three-dimensional features in the form of raised areas 351′ and recesses 353′. Each raised area 351′ may comprise curved sidewalls 371′ and an upper portion defined by a substantially planar outer portion 361′, in which the sidewalls 371′ project substantially outwardly in a direction away from the absorbent core 330 and the plane P2′ to the substantially planar outer portion 361′. In other aspects, the sidewalls 371′ defining the raised areas 351′ may be substantially linear. The planar outer portion 361′ on each raised area 351′ may comprise a substantially central location comprising an upper center point or an upper center section including the upper center point. Substantially any point on the raised area planar outer portion 361′ may define a crest or apex, which may comprise a highest point of the raised area 351′.

The recesses 353′ are adjacent to and located between the raised areas 351′ and share topsheet sidewalls 371′ with adjacent raised areas 351′. The recesses 353′ extend in a direction toward the absorbent core 330 and the plane P2′. Hence, each recess 351′ comprises curved sidewalls 371′ as well as a trough or a base defined by a substantially planar portion 363′. The substantially planar portion 363′ of each recess 351′ comprises a substantially central location comprising a lower center point or a lower center section including the lower center point. Substantially any point on the recess planar portion or base 363′ may comprise a lowest point of the recess 353′. Depending upon the size of the raised areas 351 and the spacing therebetween, the recesses 353′ may form a substantially concave outer portion. Also, the topsheet 326 may have substantially planar sections (similar to the substantially planar sections 369 of FIG. 19) located between the raised areas 351′ and the recesses 353′.

A void 359′ may be defined in the raised areas 351′ intermediate a garment-facing surface of the topsheet 326 and a substantially planar area 337A defined by a wearer-facing surface of the generally planar second material 337. The voids 359′ may provide generally the same benefits described above with respect to the voids 259 of the example of FIGS. 16 and 17A-17D. The topsheet 326 may be joined to the second material 337 in the recesses 353′, e.g., at the bases, such as by any of the methods/mechanisms discussed above for joining nonwoven layers together. In some aspects, the topsheet 326 may also be joined to the second material 337 at the generally planar sections 369.

Similar to the example depicted in FIG. 20, a width or circumference of each of the raised areas 351′ of FIG. 21 may be greatest at a point nearest the plane P2′.

A first aperture 375′ may be formed in the central location of the planar outer portion 361′, e.g., at the upper center point, of each of at least a majority of the raised areas 351′, and a second aperture 377′ may be formed in the central location of the planar portion or base 363′, e.g., at the lower center point, of each of at least a majority of the recesses 353′. The first apertures 375′ formed in the raised areas 351′ extend only through the topsheet 326, and the second apertures 377′ formed in the recesses 353′ extend through both the topsheet 326 and the second material 337.

The areas or portions of the raised areas 351′ and the recesses 353′ intermediate the apertures 375′, 377′ may be free of any additional aperture(s). For example, as seen in FIG. 21, the sidewalls 371′ of the raised areas 351′ and the recesses 353′ extending between the apertures 375′, 377′ may be continuous and contain no additional apertures. The benefits of the multi-layer material 300′ are substantially the same as those described above with respect to the multi-layer material 200 of the example of FIGS. 16 and 17A-17D. In further aspects, the raised areas 351′ and/or recesses 353′ may each comprise two or more apertures. In yet further aspects, the raised areas 351′ may be continuous, i.e., free of apertures.

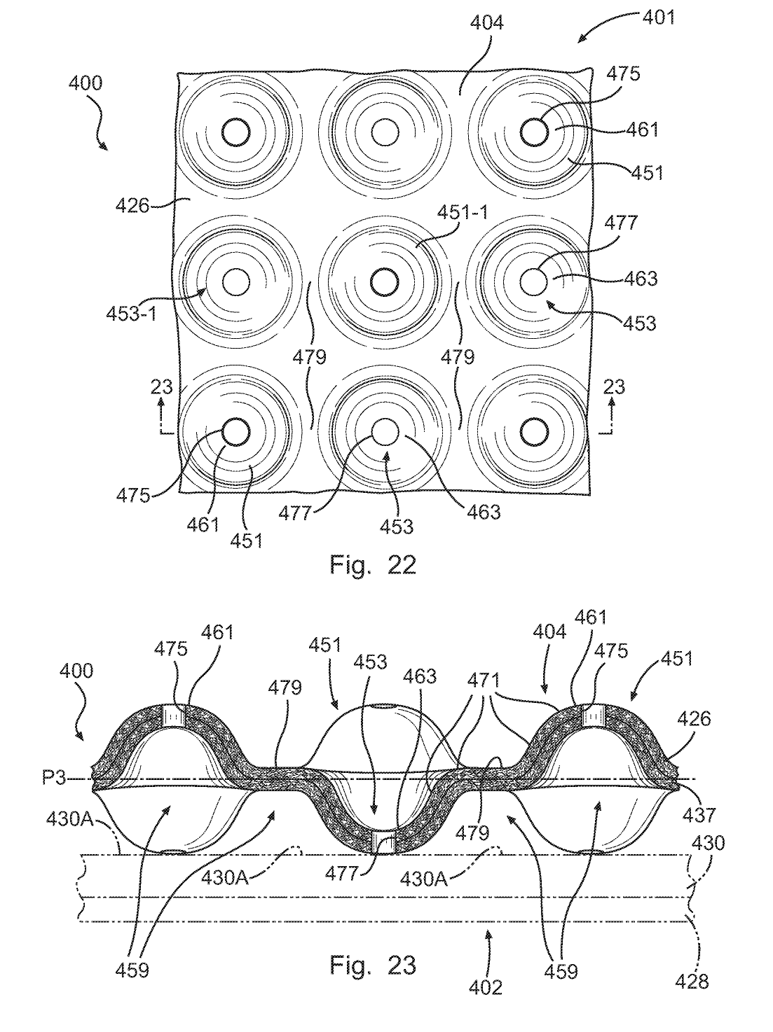

FIG. 22 is a plan view of a portion of a further example absorbent article 401 comprising a multi-layer material 400 according to the present disclosure, in which a wearer-facing surface 404 of the absorbent article is facing the viewer. FIG. 23 is a cross-sectional view of the absorbent article of FIG. 22 taken along line 23-23. The multi-layer material 400 may comprise a liquid permeable topsheet 426 and a second material 437. The absorbent article 401 may comprise the multi-layer material 400, a liquid impermeable backsheet 428 and an absorbent core 430 positioned at least partially intermediate the second material 437 and the backsheet 428. The second material 437 may be positioned intermediate the topsheet 426 and the absorbent core 430. The multi-layer material 400 may also comprise an outer cover material or other absorbent article components, as mentioned above. The topsheet 426 and second material 437 may be formed from the same nonwoven web materials used to form the topsheet 226 and second material 237 of the example of FIGS. 16 and 17A-17D described above. Further, a distribution layer may be positioned intermediate the second material 437 and the absorbent core 430. This distribution layer may comprise cross-linked cellulosic fibers, for example.

As shown in FIG. 23, the topsheet 426 may be nested with the second material 437 to form a nested laminate comprising a plurality of three-dimensional features in the form of raised areas 451 and recesses 453. The raised areas 451 may comprise curved sidewalls 471 extending upward in a direction away from the absorbent core 430 and forming a substantially convex outer portion. The raised areas 451 may be dome shaped (i.e., shaped similar to a partial sphere) as shown in FIG. 23, and in other aspects, the raised areas 451 may have a parabolic shape in cross section, a substantially conical or frustoconical shape, or any other curvilinear shape or configuration. Each of the recesses 453 may comprise curved sidewalls 471 extending downward in a direction toward the absorbent core 430 and forming a substantially concave outer portion. The recesses 453 may be dome shaped (i.e., shaped similar to a partial sphere and inverted relative to the raised areas 251) as shown in FIG. 23, and in other aspects (not show), the recesses 453 may have a parabolic shape in cross section, a substantially conical or frustoconical shape, or any other curvilinear shape or configuration. In some aspects, one or more sections of the sidewalls 471 defining the raised areas 451 and/or the recesses 453 may be substantially linear.

As shown in FIGS. 22 and 23, a substantially planar section 479 of the nested laminate may be located between adjacent raised areas 451 and recesses 453, e.g., a substantially planar section 479 is provided between raised area 451-1 and recess 453-1, to define a substantially planar stepped or spacer portion between the raised areas 451 and the recesses 453. The substantially planar sections 479 may be positioned in a plane P3 located generally between the raised areas 451 and the recesses 453. The raised areas 451 and recesses 453 extend respectively above and below the plane P3.

The three-dimensional features, i.e., the raised areas 451 and recesses 453, may be disposed in any suitable density across the surface of the nested laminate. The features may, for example, be present in a density of: from about 5 to about 100 features; alternatively, from about 10 to about 50 features; alternatively, from about 20 to about 40 features, in an area of 10 cm².

An upper portion of each raised area 451 may define a substantially central location 461 comprising a highest point or a highest section including the highest point of the raised area 451, i.e. furthest from the plane P3. The highest point may also be referred to as a crest or apex. Each recess 453 may comprise a trough or base defining a substantially central location 463 comprising a lowest point or a lowest section including the lowest point of the recess 453. Thus, the central locations 461 of the raised areas 451 may each comprise the point or section that is positioned most distal from the central locations or bases 463 of the recesses 453, and the central locations or bases 463 of the recesses 453 may each comprise the point or section that is positioned most distal from the central locations 461 of the raised areas 451. Reference may also be made to the plane P3, in which the central location 461 of each raised area 451 comprises the raised area point or section that is positioned most distal relative to the plane P3, and the central location 463 of each recess 453 comprises the recess point or area that is positioned most distal relative to the plane P3.

A void 459 may be defined between each raised area 451 and a substantially planar area 430A under the second material 437, which planar area 430A may be defined in the FIG. 23 example by the wearer-facing surface of a substantially planar region of the absorbent core 430. For example, the voids 459 may be defined intermediate a garment-facing surface of the second material 437 of the raised areas 451 and a wearer-facing surface of the substantially planar region of the absorbent core 430, as shown in the example illustrated in FIG. 23. These voids 459 may provide the same benefits described above with respect to the voids 259 of the example of FIGS. 16 and 17A-17D.

In some examples such as the FIG. 23 example, the nested laminate comprising the topsheet 426 and the second material 437 may be joined to the next layer of material toward the garment-facing surface 402 of the material 400, e.g., the absorbent core 430, only at the bases 463 of the recesses 453.

A width or circumference of each of the raised areas 451 and recesses 453 may be greatest at a point nearest the plane P3.

A first aperture 475 may be formed in the substantially central location 461, e.g., at the highest point, of each of at least a majority of the raised areas 451, and a second aperture 477 may be formed in the substantially central location or base 463, e.g., at the lowest point, of each of at least a majority of the recesses 453, as seen in FIG. 23. In the example shown in FIG. 23, the apertures 475, 477 extend through both the topsheet 426 and the second material 437. The first apertures 475 formed in the raised areas 451 may be in fluid communication with the voids 459. The apertures 475 and 477 may be shaped and sized in the same manner as described above with regards to the apertures 275, 277 in the example of FIGS. 16 and 17A-17D.

The central locations 461 and 463 of the raised areas 451 and recesses 453 may each comprise one respective aperture 475, 477, and the remaining areas or portions of the raised areas 451 and the recesses 453 may be free of any additional aperture(s). For example, as seen in FIG. 23, the sidewalls 471 of the raised areas 451 and the recesses 453 extending to the central locations 461 and 463 comprising the apertures 475, 477 may be continuous and contain no additional apertures. It is believed that by forming the raised areas 451 and the recesses 453 without apertures in the sidewalls, the strength of the raised areas 451 and recesses 453 is enhanced so as to reduce the likelihood that the raised areas 451 and/or recesses 453 may collapse under compression.

The raised areas and recesses in the example of FIGS. 22 and 23 may be modified so as to have substantially planar outer portions, similar to the planar outer portions 261′ and 263′ of the FIG. 18 example.

The example of FIGS. 22 and 23 may be further modified by providing one or more additional apertures in the raised areas 451 and/or recesses 453 and/or one or more apertures in the planar sections 479 between the raised areas 451 and the recesses 453 to further enhance movement of BM or other bodily fluids toward the absorbent core 430. In other aspects, some of the raised areas 451 and the recesses 453 may be continuous, i.e., free of apertures. The benefits of the multi-layer material 400 of the example of FIGS. 22 and 23 are substantially the same as those described above with respect to the multi-layer material 200 of the example of FIGS. 16 and 17A-17D.

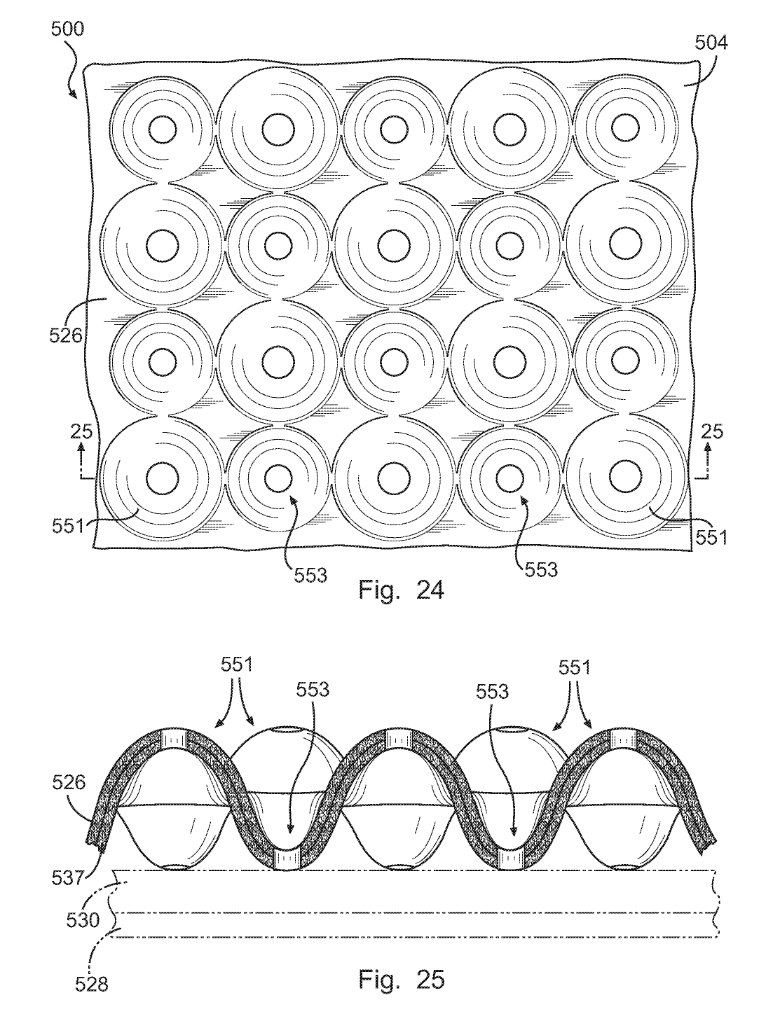

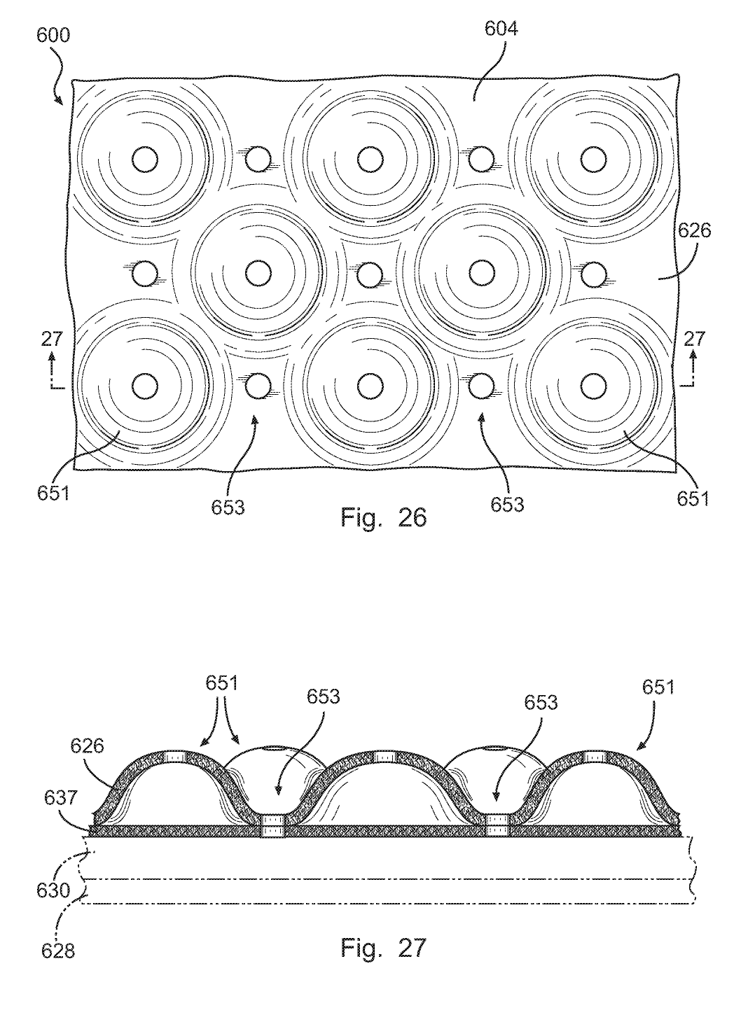

With reference now to the example of FIGS. 24 and 25, raised areas 551, which may be similar to the raised areas 251, 251′ of the examples of FIGS. 16, 17A, and 18, may form more than 50% of a total area of the topsheet 526. Recesses 553, which may be similar to the recesses 253 and 253′ in the examples of FIGS. 16, 17A and 18, are also illustrated in FIGS. 24 and 25. With reference to the example of FIGS. 26 and 27, raised areas 651, which may be similar to the raised areas 351, 351′ of the examples of FIGS. 19-21, may form more than 50% of a total area of the topsheet 626. Recesses 653, which may be similar to the recesses 353 and 353′ in the examples of FIGS. 19-21, are also illustrated in FIGS. 26 and 27. It is also contemplated that the raised areas 451 of the example of FIGS. 22 and 23 may form more than 50% of a total area of the topsheet 426.

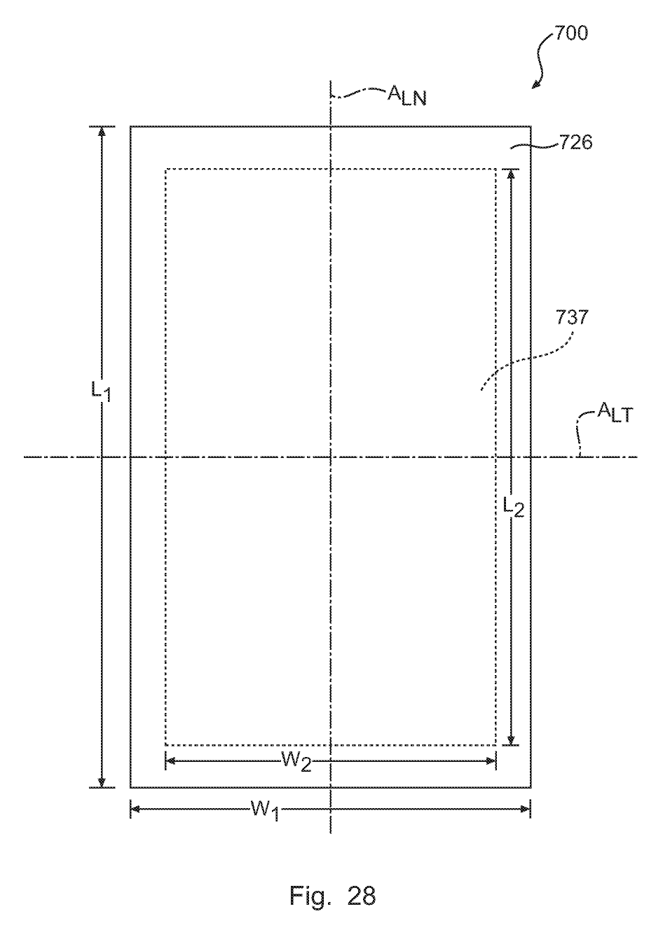

As shown in FIG. 28, an absorbent article 700 comprising a multi-layer material according to the present disclosure may comprise a central lateral axis A_{LT} and a central longitudinal axis A_{LN}. A topsheet 726 of the absorbent article 700 may comprise a first width W₁ in a direction parallel to the central lateral axis A_{LT} and a second material 737 may comprise a second width W₂ in the direction parallel to the central lateral axis A_{LT}, in which the first width W₁ is greater than the second width W₂. Also as shown in FIG. 28, the topsheet 726 may comprise a first length L₁ in a direction parallel to the central longitudinal axis A_{LN}, and the second material 737 may comprise a second length L₂ in the direction parallel to the central longitudinal axis A_{LN}, in which the first length L₁ is greater than the second length L₂. In other examples, the portion of the multi-layer material 200, 300, 400 comprising the raised areas 251, 351, 451 and recesses 253, 353, 453 may be free of continuous ridges and continuous grooves. For example, as shown in FIGS. 16, 19, and 22, each raised area 251, 351, 451 is discrete and is not continuous with neighboring raised areas 251, 351, 451. Each recess 253, 353, 453 is also discrete and is not continuous with neighboring recesses 253, 353, 453.

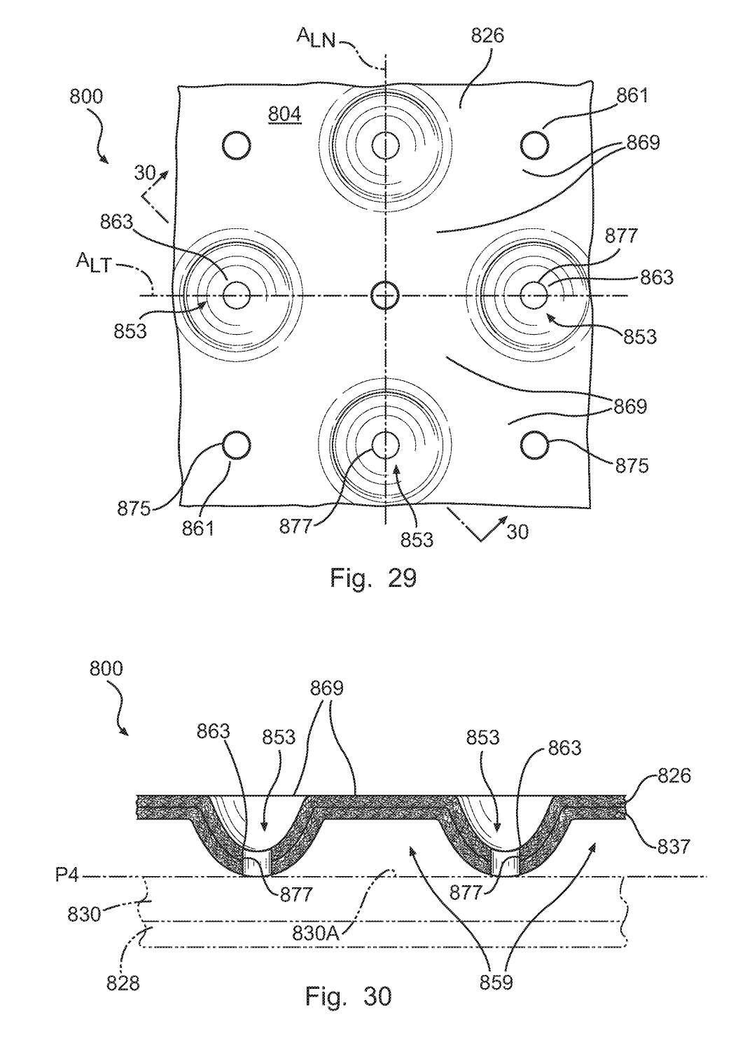

FIG. 29 is a plan view of a portion of a further example absorbent article comprising a multi-layer material 800 according to the present disclosure, in which a wearer-facing surface 804 of the absorbent article is facing the viewer. FIG. 30 is a cross-sectional view of the absorbent article of FIG. 29 taken along line 30-30. The multi-layer material 800 may comprise a liquid permeable topsheet 826 and a second material 837 that may be a separate material from the topsheet 826. The absorbent article may comprise the multi-layer material 800, a liquid impermeable backsheet 828, and an absorbent core 830 positioned at least partially intermediate the second material 837 and the backsheet 828. The second material 837 may be positioned intermediate the topsheet 826 and the absorbent core 830. The topsheet 826 and second material 837 may be formed from the same nonwoven web materials used to form the topsheet 226 and second material 237 of the example of FIGS. 16 and 17A-17D described above, and the absorbent article may comprise a similar composition and arrangement of layers as described with regards to any of the previous examples.

In the example shown in FIG. 30, the topsheet 826 may be nested with the second material 837 to form a nested laminate comprising a plurality of planar sections 869 and a plurality of discrete recesses 853. The recesses 853 may be formed by deforming a substantially planar web of the multi-layer material 800 at predefined locations in a direction away from an initial plane of the web. As shown in FIG. 30, the planar sections 869 may generally be positioned in a plane parallel to and above a plane P4 defined by the substantially planar absorbent core 830, and the recesses 853 extend in a direction toward the absorbent core 830 and the plane P4.

The recesses 853 may be shaped and sized in the same manner as described above with regards to any of the previous examples and may be formed in the same manner and with a similar density. In the example shown in FIG. 30, the recesses 853 may form a substantially concave outer portion and may each comprise a trough or base defining a substantially central location 863 comprising a lowest point or lowest section including the lowest point of the recess 853. The planar sections 869 may comprise a substantially central location 861 comprising an upper center point or an upper center section including the upper center point. The substantially central locations 861 of the planar sections 869 may be defined with respect to the substantially central locations or bases 863 of adjacent recesses 853. Substantially any point on the planar sections 869 may define a crest or apex, which may comprise a highest point on the planar section 869, e.g., furthest from the plane P4. The nested laminate comprising the topsheet 826 and the second material 837 may be joined to the next layer of material, e.g., the absorbent core 830, only at the bases of the recesses 853.

A void volume 859 may be defined intermediate a garment-facing surface of the second material 837 of the planar sections 869 and a substantially planar region of an adjacent layer of material. In the FIG. 30 example, the substantially planar region is defined by a substantially planar area 830A of a wearer-facing surface of the absorbent core 830. As shown in FIG. 29, one or more sets of the planar sections 869 defined between the discrete recesses 853 may be continuous with each other and may generally align along one or more corresponding diagonals such that each set of planar sections 869 aligned along a particular diagonal, e.g., a line extending diagonally from the left corner to the right corner in FIG. 29, may form a substantially continuous void volume 859 under the set of aligned planar sections 869.

A first aperture 875 may be formed in the substantially central location 861 of at least a majority of the planar sections 869. In the example shown in FIG. 30, the first apertures 875 may extend through both the topsheet 826 and the second material 837. The first apertures 875 may be in fluid communication with the void volume 859 defined under the planar sections 869. A second aperture 877 may optionally be formed in the substantially central locations 863 of at least a majority of the recesses 853. In the example shown in FIG. 30, the second apertures 877 may extend through both the topsheet 826 and the second material 837.

The apertures 875, 877 may be shaped and sized in the same manner as described above with regards to the apertures 275, 277 in the example of FIGS. 16 and 17A-17D. As best seen in FIG. 29, the first apertures 875 may be located in the planar sections 869 such that the first apertures 875 alternate in rows (defined along a lateral axis A_{LT}) and columns (defined along a longitudinal axis A_{LN}) with the discrete recesses 853. In particular, one or more sets of a plurality of the first apertures 875 may be generally aligned in one or more corresponding diagonals to the rows and the columns, and one or more sets of a plurality of the second apertures 877 may be generally aligned in one or more corresponding diagonals to the rows and the columns such that the first and second apertures 875, 877 alternate in rows and columns.

In other examples, the second material 837 may comprise a generally planar material, and the topsheet 826 may comprise the plurality of planar sections 869 and discrete recesses 853 such that the void volume 859 is defined between a garment-facing surface of the topsheet 826 and a wearer-facing surface of the generally planar second material 837 and the first aperture 877 formed in the planar sections 869 extends only through the topsheet 826, see FIG. 20. In further examples, the planar sections 869 may comprise additional apertures located, for example, along one or more of the corresponding diagonals.

Examples of Performance with Apertures

A few different topsheet/acquisition layer (TS/AQL) laminates were tested according to the Roll Test procedure below. Each of the TS/AQL laminate samples (labeled as “codes” below) was tested in such procedure in combination with a 222 gsm cross-linked cellulosic fiber layer glued to an 8 gsm SMS (Spunbond-Meltblown-Spunbond) support layer. Cross-linked cellulosic fiber layers have been used in disposable diapers as part of an acquisition/distribution system, for example, U.S. Pat. Publ. No. 2008/0312622 A1 to Hundorf. The TS/AQL laminate is placed with the AQL side facing the cellulosic fiber layer. The TS/AQL laminate is positioned on the cellulosic fiber layer such that it is centered over both a central lateral axis of the cellulosic fiber layer and a central longitudinal axis of the cellulosic fiber layer. The other side of the cellulosic fiber layer is facing the 8 gsm SMS support layer. The support layer is facing a flat board, such that the entire composite is on the flat board. The laminate is then secured on the board via lateral hooks present on the sides of the board. The TS/AQL laminate was 380 mm long and 180 mm wide and the AQL was 90 mm wide. The cellulosic fiber layer was 235 mm long and 80 mm wide and had a density of ca. 0.05 g/cm³.

The test fluid is a solution made with 0.5% by weight Carbopol, 5% by weight 1M NaOH solution, 95.4% by weight deionized water.

After the laminate is set up and secured to the board, 5+/−0.01 grams of test fluid are gently and uniformly applied via a syringe onto the topsheet in an area which is 20 mm wide (in a direction parallel to a central lateral axis of the TS/AQL laminate) and 60 mm long (in a direction parallel to a central longitudinal axis of the TS/AQL laminate). The area has 10 mm on each side of the central longitudinal axis of the TS/AQL laminate. The 60 mm length begins at end edge of the cellulosic fiber layer and continues 60 mm toward the other end edge of the cellulosic fiber layer. One minute after the application of the test fluid, a Plexiglas roll, having a diameter of ca. 100 mm, a width of ca. 95 mm, and a weight of 1100 g, is rolled one time over the test fluid without exerting extra pressure to the roll until reaching the opposite end of the TS/AQL laminate material along the central longitudinal axis. The roll is covered with a collagen layer via double sided adhesive tape, wherein the collagen layer is replaced after each replicate of the test.

The TS/AQL laminate and the cellulosic fiber layer are weighed prior to the rolling and after the rolling. The difference between the cellulosic fiber layer's weight after the rolling and the cellulosic fiber layer's weight prior to rolling represents the amount of test fluid that is absorbed into the cellulosic fiber layer (CABS). A higher value of CABS is desired as in fact it means that there is less fluid present over and within the TS/AQL laminate: as the test fluid is a proxy for runny BM of babies, in an in-use situation, this would mean less runny BM closer to the skin of the baby.

### EXAMPLES

### Comparative Examples

Code 1: Pattern of FIG. 6 of P&G Docket No. 14243MQ, filed on Mar. 9, 2017, of a bicomponent 20 gsm spunbond topsheet and 65 gsm carded airthrough bonded AQL without apertures.
Code 2: Pattern of FIG. 6 of P&G Docket No. 14243MQ, filed on Mar. 9, 2017, of a bicomponent 20 gsm spunbond topsheet and 65 gsm carded airthrough bonded AQL with apertures only at the bottom of discrete integral second regions (as described in P&G Docket No. 14243MQ, filed on Mar. 9, 2017), apertures 1.75 mm diameter, 5.6% effective open area (created with a hole punch).
Code 3: Pattern of FIG. 6 of P&G Docket No. 14243MQ, filed on Mar. 9, 2017, of a bicomponent 20 gsm spunbond topsheet and 65 gsm carded airthrough bonded AQL with apertures only in the generally planar first regions (as described in P&G Docket No. 14243MQ, filed on Mar. 9, 2017), apertures 1.75 mm diameter, 5.6% effective open area.

### Present Disclosure Example

Code 4: Pattern of FIG. 6 of P&G Docket No. 14243MQ, filed on Mar. 9, 2017, of U.S. Provisional Patent Application No. of a bicomponent 20 gsm spunbond topsheet and 65 gsm carded airthrough bonded AQL with apertures both at bottom of the plurality of discrete integral second regions (as described in P&G Docket No. 14243MQ, filed on Mar. 9, 2017) and in the generally planar first region (as described in P&G Docket No. 14243MQ, filed on Mar. 9, 2017), apertures 1.75 mm diameter, 11.1% effective open area.

### Data

### Amount of Test Fluid Absorbed in the Cellulosic Fiber Layer (CABS)

| | | Standard | |
|---|---|---|---|
| Code | Average, g | Deviation, g | N |
| Code 1 | 0.19 | 0.09 | 3 |
| Code 2 | 0.64 | 0.07 | 3 |
| Code 3 | 1.20 | 0.11 | 3 |
| Code 4 | 1.63 | 0.03 | 2 |

Where N is the number of replicates.

As can be seen, the code (Code 4) containing apertures in the generally planar first region and the discrete integral second regions absorbed more fluid into the cellulosic fiber layer than the non-apertured code (Code 1) and the codes (Codes 2 and 3) with apertures in only one of the generally planar first region or the discrete integral second regions.

### X. Examples of Aperture Sizes

Some example aperture sizes were determined in a nonwoven two layer web of the present disclosure in the generally planar first region (as described in P&G Docket No. 14243MQ, filed on Mar. 9, 2017) and in the plurality of discrete integral second regions (as described in P&G Docket No. 14243MQ, filed on Mar. 9, 2017). The apertures in the generally planar first regions were considerable smaller than the apertures in the discrete integral second regions owing to the deformation process (e.g., FIG. 21). First the method measuring the apertures is described.

### Aperture Measurement Method Using High Resolution MicroCT

Sample Preparation and MicroCT Scanning
A 16 mm punch is used to physically extract a representative region of the two layer web. The 16 mm diameter sample is then placed in a sample holder with an inner diameter of 17 mm. The sample is packed in super low absorbing packing material to prevent motion during the scan. The sample holder is then placed in a Scanco mCT50 x-ray scanner (Scanco Medical, Zurich, Switzerland). The scanning was performed with an energy of 45 KeV, with 3000 projections and an integration time of 5 seconds per projection. The resulting data set is 5126×5126×1355 voxels with attenuation values represented as 16 bit integers. Each voxel has a diameter of 4 microns. The file is of a proprietary format and is referred to as the ISQ file in the following steps.

Image Visualization and Analysis
The objective of the image analysis is to measure the perceived area of apertures found in the sidewalls of the depressions of the scanned two layer web samples. The ISQ files described above, were read into Avizo 9.2.0 (FEI, Houston, Tex.). The data was resampled to 8 micron voxels for easier visualization through 3D volume rendering. Upon inspection of the 3D data, 3 different apertures were identified along the sidewalls of the depressions in the two layer web. For each of these apertures, a small subvolume was created and visualized with Avizo's Volume Rendering Module. A scalebar was also added to the image for reference. The camera position of the Volume Rendering was then adjusted so that it was normal to the aperture under inspection. The viewer was set to Orthographic mode so there would not be perspective distortion in the visualization. Once the best view of the aperture is obtained, a digital image of that view is created. In addition to those apertures in the sidewall, these steps were also repeated for apertures that were not in the sidewalls of the depression for comparison.
To measure the area of the apertures from the images, we employed software developed for P&G that allows exact web based measures to be made on images. The scale bar present in the image is used to calibrate lengths or areas measured in the images. A polygonal measuring tool is used to manually create a polygon around the perimeter of the apertures and allows automatic calculation of area and perimeter.

### Sample 1

Sample 1 was produced by first overbonding a 25 gsm PE/PP spunbond bicomponent layer, laminating that layer to a layer of 65 gsm carded, through-air bonded PE/PET nonwoven with a spiral glue pattern, and then passing the laminate through a pair of rolls, as illustrated in FIG. 21, at 0.135″ (3.38 mm) engagement of the rolls. The spunbond layer was against the male roll and the carded layer was against the female roll. The laminate may represent a topsheet and an acquisition layer in an absorbent article context. Note that the overbonds were only present in the spunbond bicomponent layer and not in the carded layer. The deformation process caused by the rolls of FIG. 21 induces strain into the laminate, which causes the overbonds to rupture and form apertures in only the spunbond layer. The amount of strain in the generally planar first regions is lower than the strain in the discrete integral second regions, which results in smaller apertures in the first regions and larger apertures in the discrete integral second regions. Smaller apertures in the first regions are desirable because smaller apertures feel softer to a wearer of an absorbent article having the laminate and are less likely to mark the skin of the wearer. Larger apertures in the discrete integral second regions are preferred because they may allow faster fluid acquisition in an absorbent article context, particularly with hydrophobic webs. Since the apertures in the discrete integral second regions do not come into contact with the skin in an absorbent article context, larger apertures in the discrete integral second regions will likely not negatively impact softness or mark the skin. Sample 1 had the nested embossing pattern of FIG. 70 of P&G Docket No. 14243MQ, filed on Mar. 9, 2017.

| | Location of | No. Apertures | Average | Standard |
|---|---|---|---|---|
| | Aperture | Measured | Area (mm2) | deviation |
| Sample 1 | In discrete | 3 | 2.76 | 0.23 |
| | integral | | | |
| | second | | | |
| | regions | | | |
| Sample 1 | In generally | 4 | 0.84 | 0.36 |
| | planar first | | | |
| | region | | | |

### Examples/Combinations 1

A. An absorbent article comprising:

a liquid permeable nonwoven topsheet;

a nonwoven second material, wherein the second material is a separate material from the topsheet;

a liquid impermeable backsheet;

an absorbent core positioned at least partially intermediate the second material and the liquid impermeable backsheet;

wherein the second material is positioned intermediate the liquid permeable topsheet and the absorbent core;

wherein the topsheet comprises a plurality of recesses and a plurality of raised areas;

wherein the second material is generally planar;

wherein portions of the recesses are joined to portions of the second material;

wherein a first aperture is formed in a substantially central location of at least a majority of the raised areas, and wherein the first aperture extends through only the topsheet;

wherein the recesses each comprise a base positioned most distal from the substantially central locations of the raised areas;

wherein a second aperture is formed in at least a majority of the bases of the recesses, and wherein the second aperture extends through both the topsheet and the second material;

wherein areas of the recesses and the raised areas intermediate the first apertures and the second apertures are free of any apertures; and

wherein a void is defined intermediate a garment-facing surface of the topsheet and a wearer-facing surface of the second material in the raised areas.

B. The absorbent article of Paragraph A, wherein the second material comprises an acquisition material.
C. The absorbent article of Paragraph A or B, wherein topsheet is free of continuous ridges and continuous grooves.
D. The absorbent article of any one of Paragraphs A-C, wherein the topsheet is hydrophobic, and wherein the second material is hydrophilic.
E. The absorbent article of any one of Paragraphs A-C, wherein the topsheet is more hydrophobic than the second material.
F. The absorbent article of any one of Paragraphs A-C, wherein the topsheet is less hydrophilic than the second material.
G. The absorbent article of any one of Paragraphs A-F, wherein the first apertures are in fluid communication with the voids.
H. The absorbent article of any one of Paragraphs A-G, wherein the first apertures and the second apertures are cylindrical or ovate.
I. The absorbent article of any one of Paragraphs A-H, wherein the first apertures and the second apertures do not form conical shapes.
J. The absorbent article of any one of Paragraphs A-I, wherein the first apertures and the second apertures have a major axis that is less than 3 mm, preferably less than 2 mm.
K. The absorbent article of any one of Paragraphs A-J, wherein the raised areas form more than 50% of a total area of the topsheet.
L. The absorbent article of any one of Paragraphs A-K, wherein one or more of the raised areas comprise one of a substantially convex outer portion or a substantially planar outer portion.

### Examples/Combinations 2

A. An absorbent article comprising:

a liquid permeable nonwoven topsheet;

a nonwoven second material, wherein the second material is a separate material from the topsheet;

a liquid impermeable backsheet;

an absorbent core positioned at least partially intermediate the second material and the liquid impermeable backsheet;

wherein the second material is positioned intermediate the topsheet and the absorbent core;

wherein the topsheet comprises a plurality of planar sections and a plurality of discrete recesses;

wherein a first aperture is formed in a substantially central location of at least a majority of the planar sections, and wherein the first aperture extends through the topsheet;

wherein the recesses each form a base;

wherein the first apertures and the discrete recesses alternate in rows and columns; and

wherein one or more sets of the planar sections generally align along one or more corresponding diagonals such that each set of planar sections aligned along a diagonal forms a substantially continuous void volume under the topsheet.

B. The absorbent article of Paragraph A, wherein the topsheet is nested with the second material to form a nested laminate, the nested laminate comprising the plurality of planar sections and the plurality of discrete recesses, wherein the first aperture extends through both the topsheet and the second material.
C. The absorbent article of Paragraph A or B, wherein:

a second aperture is formed in at least a majority of the bases of the recesses and extends through both the topsheet and the second material;

one or more sets of a plurality of the first apertures are generally aligned in one or more corresponding diagonals to the rows and the columns; and

one or more sets of a plurality of the second apertures are generally aligned in one or more corresponding diagonals to the rows and the columns such that the first and second apertures alternate in rows and columns.

### Test Methods

Unless indicated otherwise, all tests described herein are made with samples conditioned at least 24 hours at 23° C.±2° C. and 50%±10% Relative Humidity (RH).

### Raised Area Factor Test Method.

### 1) General Information

The Raised Area Factor of the three-dimensional protrusions, e.g., the raised areas described herein, of the topsheet/second material laminate of an absorbent article are measured using a GFM Primos Optical Profiler instrument commercially available from GFMesstechnik GmbH, Warthestraβe 21, D14513 Teltow/Berlin, Germany. Alternative suitable non-touching surface topology profilers having similar principles of measurement and analysis, may also be used; here GFM Primos is exemplified.

The GFM Primos Optical Profiler instrument includes a compact optical measuring sensor based on a digital micro mirror projection, consisting of the following main components:

a) DMD projector with 800×600 direct digital controlled micro-mirrors
b) CCD camera with high resolution (640×480 pixels)
c) Projection optics adapted to a measuring area of at least 30×40 mm
d) Recording optics adapted to a measuring area of at least 30×40 mm
e) A table tripod based on a small hard stone plate
f) A cold light source (an appropriate unit is the KL 1500 LCD, Schott North America, Inc., Southbridge, Mass.)
g) A measuring, control, and evaluation computer running ODSCAD 6.3 software

Turn on the cold-light source. The settings on the cold-light source are set to provide a color temperature of at least 2800K.

Turn on the computer, monitor, and open the image acquisition/analysis software. In the Primos Optical Profiler instrument, select “Start Measurement” icon from the ODSCAD 6.3 task bar and then click the “Live Image button”.

The instrument is calibrated according to manufacturer's specifications using calibration plates for lateral (X-Y) and vertical (Z). Such Calibration is performed using a rigid solid plate of any non-shiny material having a length of 11 cm, a width of 8 cm and a height of 1 cm. This plate has a groove or machined channel having a rectangular cross-section, a length of 11 cm, a width of 6.000 mm and an exact depth of 2.940 mm. This groove is parallel to the plate length direction. After calibration, the instrument must be able to measure the width and depth dimensions of the groove to within ±0.004 mm.

### 2) The Raised Area Factor

The absorbent article comprising the topsheet/second material laminate with three-dimensional protrusions or raised areas, i.e., corresponding to the sample (conditioned at a temperature of 23° C.±2° C. and a relative humidity of 50%±10% for at least 24 hours) is laid down on a hard flat horizontal surface with the wearer-facing surface upward, i.e., the topsheet of the topsheet/second material laminate facing up.

Ensure that the sample is lying in planar configuration, without being stretched, with the topsheet/second material laminate uncovered. If the absorbent article features cuff and/or leg elastics, they may be carefully removed from the absorbent article by aid of scissors to eliminate any tension in the absorbent article. A nominal external pressure of 1.86 kPa (0.27 psi) is then applied to the sample. Such nominal external pressure is applied without interfering with the topology profile measurement. Such an external pressure is applied using a transparent, non-shining flat Plexiglas® plate 200 mm by 70 mm and appropriate thickness (approximately 5 mm) to achieve a weight of 83 g. The plate is gently placed on top of the sample, such that the center point of the Plexiglas® plate is at least 40 mm away from any folds, with the entire plate resting on the sample. A fold corresponds to a part of the absorbent article (e.g. the topsheet/second material laminate) where the absorbent article has been folded for packaging purposes.

Two 50 mm×70 mm metal weights each having a mass of 1200 g (approximate thickness of 43 mm) are gently placed on the Plexiglas® plate such that a 70 mm edge of each metal weight is aligned with the 70 mm edges of the Plexiglas® plate. A metal frame having external dimensions of 70 mm×80 mm and interior dimensions of 42 mm×61 mm, and a total weight of 142 g (approximate thickness 6 mm), is positioned in the center of the Plexiglas® plate between the two end weights with the longest sides of the frame aligned with the longest sides of the plate.

If the topsheet/second material laminate is smaller than 70×200 mm, or if a large enough area without a fold is not present, or if an area of interest is close to the edges of the topsheet/second material laminate and cannot be analyzed with the Plexiglas® and weights settings described above, then the X-Y dimensions of the Plexiglas® plate and the added metal weights will be adjusted to reach a nominal external pressure of 1.86 kPa (0.27 psi) while maintaining a minimum 30×40 mm field of view. At least 10 complete three-dimensional protrusions on the sample should be captured in the field of view of 30 mm×40 mm.

Position the projection head to be normal to the sample surface (i.e. the topsheet of the topsheet/second material laminate).

Adjust the distance between the sample and the projection head for best focus.

In the Primos Optical Profiler instrument, turn on the button “Pattern” to make a red cross appear on the screen cross and a black cross appears on the sample.

Adjust the focus control until the black cross is aligned with the red cross on the screen.

Adjust image brightness then capture a digitized image.

In the Primos Optical Profiler instrument, change the aperture on the lens through the hole in the side of the projector head and/or altering the camera “gain” setting on the screen.

When the illumination is optimum, the red circle at the bottom of the screen labeled “I.O.” will turn green.

Click on the “Measure” button.

The topology of the upper surface of the topsheet/second material laminate sample is measured through the Plexiglas® plate over the entire field of view 30 mm×40 mm. It is important to keep the sample still during this time in order to avoid blurring of the captured image. The image should be captured within the 30 seconds following the placement of the Plexiglas® plate, metal weights, and frame on top of the specimen.

After the image has been captured, the X-Y-Z coordinates of every pixel of the 40 mm×30 mm field of view area are recorded. The X direction is the direction parallel to the longest edge of the rectangular field of view, the Y direction is the direction parallel to the shortest edge of the rectangular field of view. The Z direction is the direction perpendicular to the X-Y plane. The X-Y plane is horizontal. These data are smoothed and filtered using a polynomial filter (n=6), a median filter 11 pixels by 11 pixels, and a structure filter 81 pixels by 81 pixels. The polynomial filter (n=6) approximates the X-Y-Z coordinate surface with a polynomial of order 6 and returns the difference to the approximated polynomial. The median filter 11 pixels by 11 pixels divides the field of view (40 mm×30 mm) in X-Y squares of 11 pixels by 11 pixels. The Z coordinate of the pixel located at the center of a given 11 pixels by 11 pixels square will be replaced by the mean Z value of all the pixels of this given square. The structure filter 81 pixels by 81 pixels, removes the waviness of the structure and translates all the Z peak values belonging to the bottom surface of the Plexiglas® plate to a top X-Y plane.

A Reference Plane is then defined as the X-Y plane intercepting the surface topology profile of the entire field of view (i.e. 30 mm×40 mm), 100 microns below this top X-Y plane. Then the Material Area of the Reference Plane is determined. The Material Area is the area of the Reference Plane that is below the surface profile. The Raised area factor is then calculated as the ratio between the Material Area of the Reference Plane and the total field of view area (i.e. 30 mm×40 mm). In the Primos Optical Profiler instrument, to measure the Material Area of the Reference Plane (Z=−0.1 mm), click on the button “Evaluate”. Then apply a pre-filtering routine including a polynomial filter (n=6), a median filter 11 by 11 and a structure filter (n=81) using the function “Filter”.

Save the image to a computer file with “.omc” extension.

Click on “Evaluate” and “Void area evaluation”.

Set the highest cutting plane to Z=0 and update the settings via clicking on “Calculate new”. Once the highest cutting plane is set to Z=0, then enter Z=−0.1 mm as the height of the Reference Plane at which the Material area will be measured and update the settings clicking on “Update”. The Material Area is then calculated.

The same above procedure set out in the Raised Area Factor is then performed on the topsheet/second material laminate with the garment-facing surface upward, i.e., the acquisition layer of the topsheet/second material laminate facing up, in which the 40 mm×30 mm field of view is located at the exact same X-Y position of the topsheet/second material laminate.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any aspect disclosed or claimed herein or that it alone, or in any combination with any other reference or references, teaches, suggests or discloses any such aspect. Further, to the extent that any meaning or definition of a term in this document conflicts with any meaning or definition of the same term in a document incorporated by reference, the meaning or definition assigned to that term in this document shall govern.

While particular aspects of the present disclosure have been illustrated and described, it would be obvious to those skilled in the art that various other changes and modifications may be made without departing from the spirit and scope of the present disclosure. It is therefore intended to cover in the appended claims all such changes and modifications that are within the scope of this disclosure.

What is claimed is:

1. An absorbent article comprising:
a liquid permeable nonwoven topsheet;
a nonwoven second material, wherein the second material is a separate material from the topsheet;
a liquid impermeable backsheet;
an absorbent core positioned at least partially intermediate the second material and the liquid impermeable backsheet;
wherein the second material is positioned intermediate the topsheet and the absorbent core;
wherein the topsheet is nested with the second material to form a nested laminate, wherein the nested laminate comprises a plurality of recesses and a plurality of raised areas, and wherein voids are defined in the raised areas under the second material;
wherein a first aperture is formed in a substantially central location of at least a majority of the raised areas, and wherein the first aperture extends through both the topsheet and the second material;
wherein the recesses each form a base positioned most distal from the substantially central locations of the raised areas;
wherein a second aperture is formed in at least a majority of the bases of the recesses, and wherein the second aperture extends through both the topsheet and the second material; and
wherein areas of the recesses and the raised areas intermediate the first apertures and the second apertures are free of any apertures.

2. The absorbent article of claim 1, wherein the second material comprises an acquisition material.

3. The absorbent article of claim 2, comprising:
a central lateral axis;
a central longitudinal axis;
wherein the topsheet comprises a first material having a first width in a direction parallel to the central lateral axis;
wherein the second material has a second width in the direction parallel to the central lateral axis; and
wherein the first width is greater than the second width.

4. The absorbent article of claim 3, wherein:
the first material has a first length in a direction parallel to the central longitudinal axis;
the second material has a second length in the direction parallel to the central longitudinal axis; and
the first length is greater than the second length.

5. The absorbent article of claim 2, wherein the acquisition material has a basis weight that is greater than a basis weight of the topsheet.

6. The absorbent article of claim 1, wherein the nested laminate is free of continuous ridges and continuous grooves.

7. The absorbent article of claim 1, wherein the first apertures are in fluid communication with the voids.

8. The absorbent article of claim 1, wherein the topsheet is hydrophobic, and wherein the second material is hydrophilic.

9. The absorbent article of claim 1, wherein the topsheet is more hydrophobic than the second material.

10. The absorbent article of claim 1, wherein the topsheet is less hydrophilic than the second material.

11. The absorbent article of claim 1, wherein the topsheet is in contact with the second material in the recesses and in the raised areas.

12. The absorbent article of claim 1, wherein the nested laminate comprises a substantially planar section extending between one of the raised areas and one of the recesses to define a stepped portion.

13. The absorbent article of claim 1, wherein the first apertures and the second apertures are cylindrical or ovate.

14. The absorbent article of claim 1, wherein the first apertures and the second apertures do not form conical shapes.

15. The absorbent article of claim 1, wherein the first apertures and the second apertures have a major axis that is less than 3 mm, preferably less than 2 mm.

16. The absorbent article of claim 1, wherein the raised areas form more than 50% of a total area of the topsheet.

17. The absorbent article of claim 1, wherein one or more of the raised areas comprise one of a substantially convex outer portion or a substantially planar outer portion.

18. The absorbent article of claim 1, wherein the voids are defined by the nested laminate raised areas and a substantially planar region of the absorbent core.

19. An absorbent article comprising:
a liquid permeable nonwoven topsheet;
a nonwoven second material, wherein the second material is a separate material from the topsheet;
a liquid impermeable backsheet;
an absorbent core positioned at least partially intermediate the second material and the liquid impermeable backsheet;
wherein the second material is positioned intermediate the liquid permeable topsheet and the absorbent core;
wherein the topsheet comprises a plurality of recesses and a plurality of raised areas;
wherein the second material is generally planar;
wherein portions of the recesses are joined to portions of the second material;
wherein a first aperture is formed in a substantially central location of at least a majority of the raised areas, and wherein the first aperture extends through only the topsheet;
wherein the recesses each comprise a base positioned most distal from the substantially central locations of the raised areas;
wherein a second aperture is formed in at least a majority of the bases of the recesses, and wherein the second aperture extends through both the topsheet and the second material;
wherein areas of the recesses and the raised areas intermediate the first apertures and the second apertures are free of any apertures; and
wherein a void is defined intermediate a garment-facing surface of the topsheet and a wearer-facing surface of the second material in the raised areas.

20. An absorbent article comprising:
a liquid permeable nonwoven topsheet;
a nonwoven second material, wherein the second material is a separate material from the topsheet;
a liquid impermeable backsheet;
an absorbent core positioned at least partially intermediate the second material and the liquid impermeable backsheet;
wherein the second material is positioned intermediate the topsheet and the absorbent core;
wherein the topsheet comprises a plurality of planar sections and a plurality of discrete recesses;
wherein a first aperture is formed in a substantially central location of at least a majority of the planar sections, and wherein the first aperture extends through the topsheet;
wherein the recesses each form a base;
wherein the first apertures and the discrete recesses alternate in rows and columns; and
wherein one or more sets of the planar sections generally align along one or more corresponding diagonals such that each set of planar sections aligned along a diagonal forms a substantially continuous void volume under the topsheet.
